(19) United States
(12) Patent Application Publication
(10) Pub. No.: US 2015/0157710 A1
(43) Pub. Date: Jun. 11, 2015

(54) DUAL OX40 AGONIST/IL-2 CANCER THERAPY METHODS

(71) Applicants: William Redmond, Hillsboro, OR (US); Andy Weinberg, Portland, OR (US)

(72) Inventors: William Redmond, Hillsboro, OR (US); Andy Weinberg, Portland, OR (US)

(73) Assignee: Providence Health & Services-Oregon d/b/a Providence Portland Medical Center, Portland, OR (US)

(21) Appl. No.: 14/381,785

(22) PCT Filed: Mar. 2, 2012
(86) PCT No.: PCT/US12/27496

§ 371 Date: Dec. 31, 2014

(51) Int. Cl. A61K 39/395; A61K 38/20

(52) U.S. Cl. CPC A61K 39/39558; A61K 38/2013; A61K 2039/505

(57) ABSTRACT

OX40 is a potent immune stimulating target. Provided herein is a method of treating cancer, which includes administering to a subject in need of treatment an OX40 agonist and a common gamma chain (yc) cytokine or an active fragment, variant, analog, or derivative thereof. In certain aspects the common gamma chain (yc) cytokine is interleukin-2 (IL-2) or an active fragment, variant, analog, or derivative thereof. Combined treatment with an agonist anti-OX40 mAb and IL-2 synergized to augment tumor immunotherapy against multiple tumor types. Dual therapy was also able to restore the function of anergic tumor-reactive CD8+ T cells.

### BACKGROUND

In addition to the classical B7-CD28 co-stimulatory pathway, recent studies have shown that members of the tumor necrosis factor receptor (TNFR) super-family, including OX40 (CD134), 4-1BB (CD137), and CD27 can augment CD4⁺ and CD8⁺ T cell responses (Watts T H, Annu Rev Immunol 2005; 23: 23-68; Croft M, Nat Rev Immunol 2003; 3: 609-20; Redmond W L and Weinberg A D, Grit Rev Immunol 2007; 27: 415-36). Specifically, work from our laboratory and others have demonstrated that OX40 ligation augments CD4⁺ and CD8⁺ T cell differentiation, cytokine production, the generation of memory T cells, and has also been shown to affect the generation and function of regulatory CD4⁺ T cells (Watts T H, Annu Rev Immunol 2005; 23: 23-68; Croft M, Annu Rev Immunol 2010; 28: 57-78; Redmond W L, et al. Crit Rev Immunol 2009; 29: 187-201). Pre-clinical studies have shown that ligation of OX40 via agonist anti-OX40 mAb, OX40L-Ig fusion proteins, or OX40L-expressing APCs can drive robust T cell-mediated anti-tumor immunity against a variety of tumors (Watts T H, Annu Rev Immunol 2005; 23: 23-68; Redmond W L and Weinberg A D, Crit Rev Immunol 2007; 27: 415-36; Croft M, Annu Rev Immunol 2010; 28: 57-78). Based upon these and other data, a phase 1 clinical trial was performed with an agonist anti-human OX40 mAb for the treatment of patients with cancer. Additional studies are underway to explore the efficacy of combining OX40-targeted therapy with other treatment modalities such as chemotherapy or radiation therapy.

One of the major advantages of targeting OX40 is the restricted nature of OX40 expression. Unlike the constitutive expression of CD28 on naïve T cells, OX40 is not expressed on naïve T cells and instead is transiently up-regulated 24-120 hours following T-cell receptor (TCR) ligation (Taraban V Y, et al. Eur J Immunol 2002; 32: 3617-27; Gramaglia I, et al. J Immunol 1998; 161: 6510-7). Previous work has shown that TCR ligation drives OX40 expression in a dose-dependent manner as stimulation with high-doses of cognate Ag was able to induce maximal OX40 expression, while weak TCR stimulation led to poor induction of OX40 (Taraban V Y, et al. Eur J Immunol 2002; 32: 3617-27; Verdeil G, et al. J Immunol 2006; 176: 4834-42). Although TCR stimulation is a necessary component for promoting the up-regulation of OX40, additional signals are required for inducing optimal OX40 expression. For example, CD28 signaling has been shown to contribute to optimal OX40-mediated co-stimulation (Walker L S, et al. J Exp Med 1999; 190: 1115-22; Rogers P R, et al. Immunity 2001; 15: 445-55), although CD28 itself is not required for the generation of OX40-dependent responses (Williams C A, et al. J Immunol 2007; 178: 7694-702; Akiba H, et al. J Immunol 1999; 162: 7058-66). Since CD28 ligation leads to increased IL-2 production and expression of the IL-2Rα (CD25) (Lenschow D J, et al. Annu Rev Immunol 1996; 14: 233-58), it is unclear whether CD28-B7 signaling contributes to OX40-mediated T cell co-stimulation directly or through an IL-2-dependent mechanism. Work from several groups has suggested that IL-2/IL-2R signaling might play a role in modulating OX40-dependent T cell co-stimulation. For example, OX40 ligation drove increased IL-2 production and CD25 expression on T cells (Gramaglia I, et al. J Immunol 2000; 165: 3043-50; Lathrop S K, et al. J Immunol 2004; 172: 6735-43 Evans D E, et al. J Immunol 2001; 167: 6804-11), while CD25-deficient T cells exhibited defective differentiation following OX40 ligation (Williams C A, et al. J Immunol 2007; 178: 7694-702; Redmond W L, et al. J Immunol 2007; 179: 7244-53). However, these studies did not address directly whether IL-2R signaling affects OX40 expression.

IL-2/IL-2R signaling occurs via the trimeric IL-2 receptor which consists of the IL-2Rα (CD25), IL-2/IL-15Rβ (CD122), and common gamma (yc; CD132) chains (Nelson B H, and Willerford D M. Adv Immunol 1998; 70: 1-81). IL-2R signaling is initiated by phosphorylation of JAK3 and JAK1, which are constitutively associated with the γc and IL-2Rβ chains, respectively. Activation of these kinases leads to the activation of several downstream molecules, including PI3K/AKT, MAPK/ERK, and the STAT family of transcription factors (Gaffen S L. Cytokine 2001; 14: 63-77). Other members of the IL-2 cytokine family also utilize the γc subunit including IL-4, IL-7, IL-9, IL-15, and IL-21. Importantly, whether IL-2R and/or common γc cytokine signaling regulates OX40 expression remains controversial. While some studies have shown that IL-2 and IL-4 can up-regulate OX40 expression on T cells, others demonstrated that IL-2R signaling was dispensable for inducing OX40 (Verdeil G, et al. J Immunol 2006; 176: 4834-42; Williams Calif., et al. J Immunol 2007; 178: 7694-702; Toennies H M, et al. J Leukoc Biol 2004; 75: 350-7).

There remains a need to develop new cancer immunotherapies and improve existing cancer immunotherapies through the OX40 pathway.

### BRIEF SUMMARY

The present disclosure demonstrates that OX40 expression is driven via a dual TCR/common γc cytokine-dependent signaling pathway, which is dependent upon activation of JAK3 and its downstream targets, the transcription factors STAT3 and STAT5. In certain aspects, the present disclosure further demonstrates that combination therapy with an OX40 agonist and IL-2 can enhance tumor regression. In other aspects the disclosure shows that dual anti-OX40/IL-2 therapy can further restore the function of anergic tumor-reactive CD8 T cells, e.g., in mice with long-term well-established tumors, leading to enhanced survival. This disclosure shows that combined anti-OX40/γc cytokine (e.g., IL-2)-directed therapy can improve tumor immunotherapy and revive the function of tumor-reactive CD8 T cells for the treatment of patients with cancer.

Provided herein is method of treating cancer which includes administering to a subject in need of treatment an OX40 agonist and a common gamma chain (γc) cytokine or an active fragment, variant, analog, or derivative thereof. In certain aspects, the administration is synergistic, i.e., it stimulates T-lymphocyte-mediated anti-cancer immunity to a greater extent than the OX40 agonist or γc cytokine alone. In certain aspects the administration stimulates T-lymphocytes, e.g., CD4⁺, CD8⁺ or both CD4⁺ and CD8⁺ T-lymphocytes. In certain aspects, the administration can restore the function of anergic tumor-reactive T-lymphocytes, e.g., CD8⁺ T-lymphocytes. In certain aspects proliferation of anergic tumor-reactive CD8 T-lymphocytes is restored, in certain aspects differentiation of the anergic tumor-reactive CD8⁺ T-lymphocytes is restored. In certain aspects both proliferation and differentiation are restored.

Further provided is a method of enhancing the effect of an OX40 agonist on T-lymphocyte-mediated cancer immunotherapy, where the method includes contacting T Cell Receptor (TCR)-stimulated T-lymphocytes with an OX40 agonist in combination with a γc cytokine, e.g., IL-2, or an active fragment, variant, analog, or derivative thereof. Another method of enhancing the effect of an OX40 agonist on T-lymphocyte-mediated cancer immunotherapy is also provided, where the method includes stimulating T-lymphocytes via TCR ligation, and contacting the TCR-stimulated T-lymphocytes with an OX40 agonist in combination with a γc cytokine, or an active fragment, variant, analog, or derivative thereof. In certain aspects of the provided methods of enhancing the effect of an OX40 agonist on T-lymphocyte-mediated cancer immunotherapy the cancer immunotherapy requires CD4⁺ T-lymphocytes, CD8⁺ T-lymphocytes or both CD4⁺ T-lymphocytes and CD8⁺ T-lymphocytes. In certain aspects the contacting can stimulate T-lymphocyte-mediated cancer immunotherapy to a greater extent than the OX40 agonist or γc cytokine alone, the contacting can restore the function of anergic tumor-reactive CD8⁺ T cells, or both.

Further provided is a method of enhancing OX40 agonist-mediated augmentation of T-lymphocyte proliferation in response to TCR stimulation, where the method includes contacting TCR-stimulated T-lymphocytes with an OX40 agonist in combination with a γc cytokine, or an active fragment, variant, analog, or derivative thereof. Another method of enhancing OX40 agonist-mediated augmentation of T-lymphocyte proliferation is also provided, where the method includes stimulating T-lymphocytes via TCR ligation, and contacting the TCR-stimulated T-lymphocytes with an OX40 agonist in combination with a γc cytokine, or an active fragment, variant, analog, or derivative thereof. In certain aspects the enhancement also includes enhancement of T-lymphocyte differentiation. In certain aspects TCR ligation is accomplished through contacting the T-lymphocytes with an antigen/MHC complex. The antigen can be, for example a cancer cell-specific antigen. In certain aspects the TCR ligation is accomplished through contacting the T-lymphocytes with anti-CD3. The anti-CD3 can be, for example, bound to a solid substrate. TCR ligation accomplished through contact with anti-CD3 can further include contacting the T-lymphocytes with anti-CD28. TCR ligation accomplished through contact with anti-CD3 can be carried out in vivo, in vitro, or ex vivo.

In certain aspects of the methods provided herein, the γc cytokine can be IL-2, IL4, IL7, IL-21, any active fragment, variant, analog or derivative thereof, or a combination thereof. In certain specific aspects the γc cytokine is IL-2 or an active fragment, variant, analog or derivative thereof, and a combination thereof. In certain aspects the IL-2 can be aldesleukin, BAY 50-4798, NHS-EMD 521873, or any combination thereof.

In certain aspects of the methods provided herein the γc cytokine upregulates OX40 expression in the T-lymphocytes. In certain aspects the upregulation can be mediated through the JAK3 phosphorylation, e.g., through JAK3 activation of STAT5, STAT3, or both STAT5 and STAT3. In specific aspects, the upregulation is mediated through JAK3 activation of STAT5.

In certain aspects of the methods provided herein the OX40 agonist is a binding molecule which specifically binds to OX40.

In certain aspects the binding molecule includes an antibody which specifically binds to OX40, or an antigen-binding fragment thereof, e.g., a monoclonal antibody, a chimeric antibody, a humanized antibody, or a human antibody. In certain aspects the antigen-binding fragment is an Fab fragment, an Fab′ fragment, an F(ab)2 fragment, a single-chain Fv fragment, or a single chain antibody. In certain aspects the antibody which specifically binds to OX40, or an antigen-binding fragment thereof binds to the same OX40 epitope as mAb 9B12.

In certain aspects the binding molecule includes an OX40 ligand or OX40-binding fragment thereof.

In certain aspects the binding molecule further includes a heterologous polypeptide fused thereto. In certain aspects the binding molecule is conjugated to an agent selected from the group consisting of a therapeutic agent, a prodrug, a peptide, a protein, an enzyme, a virus, a lipid, a biological response modifier, a pharmaceutical agent, or PEG.

In certain aspects the binding molecule includes a fusion polypeptide, including in an N-terminal to C-terminal direction: an immunoglobulin domain, wherein the immunoglobulin domain includes an Fc domain; a trimerization domain, wherein the trimerization domain includes a coiled coil trimerization domain; and a receptor binding domain, wherein the receptor binding domain is an OX40 receptor binding domain, and wherein the fusion polypeptide self-assembles into a trimeric fusion protein. In certain aspects this fusion polypeptide is capable of binding to the OX40 receptor and stimulating at least one OX40 mediated activity. In certain aspects this the OX40 receptor binding domain of this fusion polypeptide includes an extracellular domain of OX40 ligand (OX40L). In certain aspects the trimerization domain of this fusion protein includes a TRAF2 trimerization domain, a Matrilin-4 trimerization domain, or a combination thereof.

In certain aspects of the methods provided herein the cancer is a solid tumor, or a metastasis thereof. In certain aspects of the methods provided herein the cancer is, for example, melanoma, gastrointestinal cancer, renal cell carcinoma, prostate cancer, lung cancer, breast cancer or any combination thereof. In certain aspects of the methods provided herein where the cancer has metastasized, a metastasis can be sited in lymph node, lung, liver, bone, or any combination thereof.

In certain aspects of the methods provided herein the treatment further includes administering to the patient at least one additional cancer treatment. The additional cancer treatment can be, for example, surgery, radiation, chemotherapy, immunotherapy, targeting anti-cancer therapy, hormone therapy, or any combination thereof.

In certain aspects of the methods provided herein the OX40 agonist is administered as a single dose. In certain aspects of the methods provided herein the yc cytokine is administered as a single dose. In certain aspects of the methods provided herein the OX40 agonist is administered in at least two doses. In certain aspects of the methods provided herein the γc cytokine is administered in at least two doses. In certain aspects of the methods provided herein the OX40 agonist is administered by IV infusion. In certain aspects of the methods provided herein the γc cytokine is administered by IV infusion.

In certain aspects of the methods provided herein, the γc cytokine can be administered to the subject prior to administration of the OX40 agonist, simultaneously with the administration of the OX40 agonist, or after administration of the OX40 agonist.

In certain aspects of the methods provided herein the subject is a human patient. In certain aspects the treatment can result in a regression of at least one tumor or metastasis in the patient, retarded or no increase in tumor or metastatic growth in the patient, stabilization of disease in the patient, prolonged survival of the patient, retardation, stalling or decrease in growth of a long-term established tumor or metastasis thereof, or any combination thereof.

### BRIEF DESCRIPTION OF THE DRAWINGS/FIGURES

FIG. 1. OX40 is regulated by the strength of TCR stimulation and IL-2Rα (CD25) expression. A) Expression of CD25 or OX40 by OT-I T cells at day 3 following TCR stimulation with APCs treated with increasing amounts of cognate peptide as indicated. Expression was measured by flow cytometry. B) Graphs showing expression kinetics of CD25 and OX40 following TCR stimulation at the indication time points, as determined by flow cytometry. (C) Graphs showing CD25 and OX40 expression levels in purified nave and carboxyfluorescein diacetate succinimidyl ester (CFSE)-labeled polyclonal wild-type or CD25−/− CD8⁺ T cells following anti-CD3 and anti-CD28 stimulation. Expression was measured by flow cytometry. *P<0.05.

FIG. 2. OX40 is regulated on murine and human T cells by TCR stimulation and IL-2. A) CD25 and OX40 expression by wild-type or OX40−/− OT-I T cells activated with peptide-pulsed APCs, and then stimulated with media alone or with recombinant murine IL-2, as determined by flow cytometry. Bar graphs depict the mean+/−SEM (n=6/group). B) CD25 and OX40 expression by human CD8⁺ T cells stimulated with recombinant human IL-2 and/or anti-CD3 mAb (OKT-3) as determined by flow cytometry. C) Bar graphs depicting CD25 and OX40 expression in human CD8⁺ and CD4⁺ T cells stimulated with media, recombinant human IL-2 and/or anti-CD3 mAb (OKT-3) as determined by flow cytometry. The data represents the mean+/− SD (n=3-5/group). Data are pooled from five independent experiments with similar results. *P<0.05; **P<0.01; ***P<0.001.

FIG. 3. Common γc cytokines regulate OX40 via JAK/STAT signaling. A) The level of phosphorylation of JAK1, JAK2, and JAK3 in stimulated WT OT-I T cells in the presence of absence of recombinant murine IL-2 assessed by Western blot. B) CD25 and OX40 expression by WT OT-I T cells activated with peptide-pulsed APCs, and then stimulated in the presence of absence of recombinant murine IL-2, when treated with a JAK3 inhibitor (PF-956980), as determined by flow cytometry. C) CD25 expression (% positive and mean fluorescence intensity (MFI)) by WT or OX40−/− OT-I cells activated with peptide-pulsed APCs, and then treated with media alone, or with recombinant murine IL-2, IL-4, IL-7, IL-9, IL-15, or IL-21, as determined by flow cytometry. D) Ox40 expression (% positive and MFI) by WT or OX40−/− OT-I cells activated with peptide-pulsed APCs, and then treated with media alone, or with recombinant murine IL-2, IL-4, IL-7, IL-9, IL-15, or IL-21, as determined by flow cytometry. E) The level of phosphorylation of STAT1, STAT5, STAT4, STAT5, and STAT6 in stimulated WT OT-I T cells in the presence of absence of recombinant murine IL-2, IL4, IL7, IL15 and IL21 assessed by Western blot. The bar graphs in B)-D) depict the mean+/−SD from B) n=2-3/group or C, D) n=3-8/group. Data are representative of one out of two to ten independent experiments with similar results. *P<0.05; **P<0.01; ***P<0.001.

FIG. 4: Induction of maximal OX40 expression by common γc cytokines is regulated by the strength of TCR stimulation. A) CD25 expression (% positive and MFI) by WT OT-I T cells activated with either wild-type (SIINFEKL) or altered peptide ligand (SIITFEKL) pOVA-pulsed APCs, followed by treatment with media alone, or with recombinant murine IL-2, IL-4, IL-7, IL-15, or IL-21, as determined by flow cytometry. B) OX40 expression (% positive and MFI) by WT OT-I T cells stimulated with either wild-type (SIINFEKL) or altered peptide ligand (SIITFEKL) pOVA-pulsed APCs, followed by treatment with media alone, or with recombinant murine IL-2, IL-4, IL-7, IL-15, or IL-21, as determined by flow cytometry. Graphs depict the mean+/−SEM from n=2/group. *P<0.001.

FIG. 5. STAT3 and STAT5 are required for optimal up-regulation of OX40 following stimulation with common γc cytokines. A) CD25 and OX40 expression (% positive and MFI) by WT OT-I T cells activated with peptide-pulsed APCs and then stimulated with media alone, or with recombinant murine IL-2, IL-4, or IL-21, as measured by flow cytometry. B) CD25 and OX40 expression (% positive and MFI) by polyclonal endogenous WT or STAT5−/− CD8⁺ T cells stimulated with anti-CD3 mAb, harvested, and then stimulated with media alone, or with recombinant murine IL-2, IL-4, or IL-21, as determined by flow cytometry. The bar graphs depict the mean+/− SD (n=2-3/group). Data are representative of one out of two independent experiments with similar results. *P<0.05; **P<0.01; ***P<0.001; NS no statistically significant difference.

FIG. 6. IL-2 treatment enhanced OX40 expression on CD8⁺ T cells in tumor-bearing hosts. The extent of CD25, YFP (OX40 reporter), and OX40 expression on CD8⁺ T cells isolated from the A) tumor and B) spleen of tumor-bearing C57BL/6 OX40-cre x ROSA-YFP reporter mice treated with IL-2 cytokine/mAb complexes, as assessed by flow cytometry. Graphs depict the results obtained from 3-4 individual animals from 1 out of 2 independent experiments with similar results.

FIG. 7. Combined anti-OX40/IL-2c therapy boosts anti-tumor immunity through a T cell-dependent mechanism. Tumor growth and survival of MCA-205 tumor-bearing wild-type mice treated with anti-OX40 or rat IgG Ab along with IL-2 cytokine/mAb complexes. The extent of A) tumor growth and B) survival of tumor-bearing mice were assessed. Data are representative of one out of 2 independent experiments with similar results. C) Survival of CD4, CD8, or CD4/CD8-depleted MCA-205 tumor-bearing mice treated with anti-OX40 and IL-2c.

FIG. 8. Treg functional assay. The effect of anti-OX40/IL-2c treatment on Treg function in tumor-bearing mice. Graphs depict the mean+/−SD from n=2-3/group.

FIG. 9. Dual anti-OX40/I-2c therapy reverses CD8 T cell anergy and increases the survival of mice with long-term well-established tumors. A) Tumor model. B) Ki-67, granzyme B, and KLRG-1 expression on donor OT-I T cells in the peripheral blood as determined by flow cytometry. C) In vivo CTL assay. D, E) The extent of tumor growth (mean+/−SD; n=5/group) and D) survival (n=11/group) of tumor-bearing mice were assessed. Data are representative of one out of 2 to 3 independent experiments with similar results or E) the cumulative survival from 2 independent experiments. *P<0.05, **P<0.01.

### DETAILED DESCRIPTION OF THE INVENTION

### I. Definitions

It is to be noted that the term “a” or “an” entity refers to one or more of that entity; for example, “an OX40 agonist” is understood to represent one or more OX40 agonists. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety.

It is understood that wherever embodiments are described herein with the language “comprising,” otherwise analogous embodiments described in terms of “consisting of” and/or “consisting essentially of” are also provided.

The terms “OX40” and “OX40 receptor” are used interchangeably herein. The receptor is also referred to as CD134, ACT-4, and ACT35. OX40 is a member of the TNFR-superfamily of receptors, and is expressed on the surface of antigen-activated mammalian CD4⁺ and CD8⁺ T-lymphocytes (Paterson, D. J., et al. Mol Immunol 24, 1281-1290 (1987); Mallett, S., et al. EMBO J 9, 1063-1068 (1990); Calderhead, D. M., et al. J Immunol 151, 5261-5271 (1993)).

As used herein, the term OX40 ligand (“OX40L”), also variously termed gp34, ACT-4-L, and CD252, is a protein that specifically interacts with the OX40 receptor (Baum P. R., et al. EMBO J. 13:3992-4001(1994)). The term OX40L includes the entire OX40 ligand, soluble OX40 ligand, and fusion proteins including a functionally active portion of OX40 ligand covalently linked to a second moiety, e.g., a protein domain. Also included within the definition of OX40L are variants which vary in amino acid sequence from naturally occurring OX4L but which retain the ability to specifically bind to the OX40 receptor. Further included within the definition of OX40L are variants which enhance the biological activity of OX40.

As used herein, an “agonist,” e.g., an OX40 agonist is a molecule which enhances the biological activity of its target, e.g., OX40. In a certain aspects blocking OX40 agonists, including, e.g., anti-OX40 antibodies or OX40 ligand compositions, substantially enhance the biological activity of OX40. Desirably, the biological activity is enhanced by 10%, 20%, 30%, 50%, 70%, 80%, 90%, 95%, or even 100%. In certain aspects, OX40 agonists as disclosed herein include OX40 binding molecules, e.g., binding polypeptides, e.g., anti-OX40 antibodies, OX40L, or fragments or derivatives of these molecules.

A “binding molecule” or “antigen binding molecule” refers in its broadest sense to a molecule that specifically binds target, e.g., OX40 receptor. In one aspect, a binding molecule is an antibody or an antigen-binding fragment thereof. In another aspect, a binding molecule includes at least one heavy or light chain CDR of a reference antibody molecule. In another aspect, a binding molecule includes at least two, three, four, five, or six CDRs from one or more reference antibody molecules.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins including an antigen determination portion of an antibody, and any other modified immunoglobulin molecule including an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

An “OX40 binding molecule” as described herein is an agent which binds substantially only to OX40 present on the surface of mammalian T-cells, such as activated CD4⁺ T-cells. As used herein, the term “OX40 binding molecule” includes anti-OX40 antibodies and OX40L.

The terms “antigen binding fragment” refers to a portion of an intact antibody and refers to the antigenic determining variable regions of an intact antibody. It is known in the art that the antigen binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

A “variable region” of an antibody refers to the variable region of the antibody light chain or the variable region of the antibody heavy chain, either alone or in combination. The variable regions of the heavy and light chain each consist of four framework regions (FW) connected by three complementarity determining regions (CDRs) also known as hypervariable regions. The CDRs in each chain are held together in close proximity by the FW regions and, with the CDRs from the other chain, contribute to the formation of the antigen-binding site of antibodies. There are at least two techniques for determining CDRs: (I) an approach based on cross-species sequence variability (i.e., Kabat et al. Sequences of Proteins of immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins including an antibody portion, and any other modified immunoglobulin molecule including an antigen recognition site. Furthermore, “monoclonal antibody” refers to such antibodies made in any number of ways including, but not limited to, by hybridoma, phage selection, recombinant expression, and transgenic animals.

The term “chimeric antibody” refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc) with the desired specificity, affinity, and functional capability while the constant regions are homologous to the sequences in antibodies derived from another (usually human) to avoid eliciting an immune response in that species.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has the desired specificity, affinity, and capability.

A humanized antibody can be further modified by the substitution of additional residues either in the Fv framework region and/or within the replaced non-human residues to refine and optimize antibody specificity, affinity, and/or capability. In general, the humanized antibody can include substantially all of at least one, and typically two or three, variable domains containing all or substantially all of the CDR regions that correspond to the non-human immunoglobulin whereas all or substantially all of the FW regions are those of a human immunoglobulin consensus sequence. A humanized antibody can also include at least a portion of an immunoglobulin constant region or domain (Fc), typically that of a human immunoglobulin. Examples of methods used to generate humanized antibodies are described in U.S. Pat. No. 5,225,539 or 5,639,641.

As used herein, “human” or “fully human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example, in U.S. Pat. No. 5,939,598 by Kucherlapati et al. “Human” or “fully human” antibodies also include antibodies including at least the variable domain of a heavy chain, or at least the variable domains of a heavy chain and a light chain, where the variable domain(s) have the amino acid sequence of human immunoglobulin variable domain(s).

“Human” or “fully human” antibodies also include antibodies that comprise, consist essentially of, or consist of, variants (including derivatives). Standard techniques known to those of skill in the art can be used to introduce mutations in the nucleotide sequence encoding a human antibody, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3.

The term “anti-OX40 antibodies” and grammatical equivalents encompasses monoclonal and polyclonal antibodies which are specific for OX40, i.e., which bind substantially only to OX40, as well as antigen-binding fragments thereof. In certain aspects, anti-OX40 antibodies as described herein are monoclonal antibodies (or antigen-binding fragments thereof), e.g., murine, humanized, or fully human monoclonal antibodies.

The term “anergy” refers to a specific kind if immune modulation, in which certain cells of the immune system are rendered non-responsive to antigen stimulus.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for cancer according to the methods described herein if the patient shows, e.g., total, partial, or transient remission of a certain type of cancer.

A subject is successfully “treated” according to the methods of described herein if the patient shows one or more of the following: a reduction in the number of or complete absence of cancer cells: a reduction in the tumor size; or retardation or reversal of tumor growth, inhibition, e.g., suppression, prevention, retardation, shrinkage, or reversal of metastases, e.g., of cancer cell infiltration into peripheral organs including, for example, the spread of cancer into soft tissue and bone; inhibition of, e.g., suppression of, retardation of, prevention of, shrinkage of, reversal of or an absence of tumor metastases; inhibition of, e.g., suppression of, retardation of, prevention of, shrinkage of, reversal of or an absence of tumor growth; relief of one or more symptoms associated with the specific cancer; reduced morbidity and mortality; improvement in quality of life; or some combination of effects. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The terms “cancer”, “tumor”, “cancerous”, and “malignant” refer to or describe the physiological condition in mammals that is typically characterized by unregulated cell growth. Examples of cancers include but are not limited to, melanoma, gastrointestinal cancer, renal cell carcinoma, prostate cancer, and lung cancer.

The terms “metastasis,” “metastases,” “metastatic,” and other grammatical equivalents as used herein refer to cancer cells which spread or transfer from the site of origin (e.g., a primary tumor) to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures. The terms also refer to the process of metastasis, which includes, but is not limited to detachment of cancer cells from a primary tumor, intravasation of the tumor cells to circulation, their survival and migration to a distant site, attachment and extravasation into a new site from the circulation, and microcolonization at the distant site, and tumor growth and development at the distant site. In certain aspects, metastases appear in sites including, but not limited to lymph node, lung, liver, and bone.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, bears, and so on.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purpose of this disclosure, as are native or recombinant polypeptides that have been separated, fractionated, or partially or substantially purified by any suitable technique.

Also included as polypeptides are fragments, derivatives, analogs, or variants of the foregoing polypeptides, and any combination thereof. The terms “fragment,” “variant,” “derivative,” and “analog” when referring, e.g., to OX40 agonist polypeptides include any polypeptides that retain at least some of the binding properties of the corresponding OX40 agonist. Fragments of polypeptides include proteolytic fragments, as well as deletion fragments, in addition to specific antibody fragments discussed elsewhere herein. As used herein a “derivative,” e.g., of an OX40 agonist polypeptide refers to a subject polypeptide having one or more residues chemically derivatized by reaction of a functional side group. Also included as “derivatives” are those peptides that contain one or more naturally occurring amino acid derivatives of the twenty standard amino acids.

The terms “T cell” and “T-lymphocyte” are used interchangeably herein to refer to the population of lymphocytes carrying a T cell receptor complex (including the T-cell-specific CD3 marker) on the cell surface. While T-lymphocytes very generally function in cell-mediated immunity, they can be divided into myriad sub-populations based not only on their particular functions, but also on the differential expression of certain surface and intracellular antigens, which can function as “markers” for particular T-lymphocyte sub-populations. As a general non-limiting example, helper T-cells express the surface antigen CD4, where cytotoxic T-cells express CD8. Sub-populations within these groups, and overlapping between these groups can be identified by other cell surface markers including, but not limited to CD95, CD25, FoxP3, CD28, CCR7, CD127, CD38, HLA-DR, and Ki-67. Subpopulations of T-lymphocytes can be identified and/or isolated from a mixed population of blood cells through the use of labeled antibodies, e.g., through flow cytometry or fluorescence activated cell sorting, described in more detail in the examples below. For example helper T cells can be identified as expressing CD3 and CD4, but not FoxP3. Other overlapping and non-overlapping subpopulations of T-lymphocytes include memory T cells, immature T cells, mature T cells, regulatory T cells (Tregs), activated T cells, and natural killer T (NKT) cells.

### II. OX40 Agonists

OX40 agonists interact with the OX40 receptor on CD4⁺ T-cells during, or shortly after, priming by an antigen results in an increased response of the CD4⁺ T-cells to the antigen. In the context of the present disclosure, the term “agonist” refers to molecules which bind to and stimulate at least one activity mediated by the OX40 receptor. For example, an OX40 agonist interacting with the OX40 receptor on antigen specific CD4⁺ T-cells can increase T cell proliferation as compared to the response to antigen alone. The elevated response to the antigen can be maintained for a period of time substantially longer than in the absence of an OX40 agonist. Thus, stimulation via an OX40 agonist enhances the antigen specific immune response by boosting T-cell recognition of antigens, e.g., tumor cells. OX40 agonists are described, for example, in U.S. Pat. Nos. 6,312,700, 7,504,101, 7,622,444, and 7,959,925, which are incorporated herein by reference in their entireties.

OX40 agonists include, but are not limited to OX40 binding molecules, e.g., binding polypeptides, e.g., OX40 ligand (“OX40L”) or an OX40-binding fragment, variant, or derivative thereof, such as soluble extracellular ligand domains and OX40L fusion proteins, and anti-OX40 antibodies (for example, monoclonal antibodies such as humanized monoclonal antibodies), or an antigen-binding fragment, variant or derivative thereof. Examples of anti-OX40 monoclonal antibodies and are described in WO 95/12673 and WO 95/21915, the disclosures of which are incorporated herein by reference in their entireties. In certain aspects, the anti-OX40 monoclonal antibody is 9B12, or an antigen-binding fragment, variant, or derivative thereof, as described in Weinberg, A. D., et al. J Immunother 29, 575-585 (2006), which is incorporated herein by reference in its entirety.

In one aspect, an OX40 agonist includes a fusion protein in which one or more domains of OX40L is covalently linked to one or more additional protein domains. Exemplary OX40L fusion proteins that can be used as OX40 agonists are described in U.S. Pat. No. 6,312,700, the disclosure of which is incorporated herein by reference in its entirety.

In one aspect, an OX40 agonist includes an OX40L fusion polypeptide that self-assembles into a multimeric (e.g., trimeric or hexameric) OX40L fusion protein. Such fusion proteins are described, e.g., in U.S. Pat. No. 7,959,925, which is incorporated by reference herein in its entirety. The multimeric OX40L fusion protein exhibits increased efficacy in enhancing antigen specific immune response in a subject, particularly a human subject, due to its ability to spontaneously assemble into highly stable trimers and hexamers.

In certain aspects, an OX40 agonist capable of assembling into a multimeric form includes a fusion polypeptide, including in an N-terminal to C-terminal direction: an immunoglobulin domain, wherein the immunoglobulin domain includes an Fc domain, a trimerization domain, wherein the trimerization domain includes a coiled coil trimerization domain, and a receptor binding domain, wherein the receptor binding domain is an OX40 receptor binding domain, e.g., an OX40L or an OX40-binding fragment, variant, or derivative thereof, where the fusion polypeptide can self-assemble into a trimeric fusion protein. In one aspect, an OX40 agonist capable of assembling into a multimeric form is capable of binding to the OX40 receptor and stimulating at least one OX40 mediated activity. In certain aspects, the OX40 agonist includes an extracellular domain of OX40 ligand.

The trimerization domain of an OX40 agonist capable of assembling into a multimeric form serves to promote self-assembly of individual OX40L fusion polypeptide molecules into a trimeric protein. Thus, an OX40L fusion polypeptide with a trimerization domain self-assembles into a trimeric OX40L fusion protein. In one aspect, the trimerization domain is an isoleucine zipper domain or other coiled coli polypeptide structure. Exemplary coiled coil trimerization domains include: TRAF2 (GENBANK® Accession No. Q12933, amino acids 299-348; Thrombospondin 1 (Accession No. PO7996, amino acids 291-314; Matrilin-4 (Accession No. 095460, amino acids 594-618); CMP (matrilin-1) (Accession No. NP002370, amino acids 463-496); HSF1 (Accession No. AAX42211, amino acids 165-191); and Cubilin (Accession No. NP001072, amino acids 104-138). In certain specific aspects, the trimerization domain includes a TRAF2 trimerization domain, a Matrilin-4 trimerization domain, or a combination thereof.

It can further be desirable to modify an OX40 agonist in order to increase its serum half-life. For example, the serum half-life of an OX40 agonist can be increased by conjugation to a heterologous molecule such as serum albumin, an antibody Fc region, or PEG. In certain embodiments, OX40 agonists can be conjugated to other therapeutic agents or toxins to form immunoconjugates and/or fusion proteins. In certain aspects, an OX40 agonist can be conjugated to an agent selected from the group that includes a therapeutic agent, a prodrug, a peptide, a protein, an enzyme, a virus, a lipid, a biological response modifier, or a pharmaceutical agent. Suitable toxins and chemotherapeutic agents are described in Remington's Pharmaceutical Sciences, 19th Ed. (Mack Publishing Co. 1995), and in Goodman and Gilman's the Pharmacological Basis of Therapeutics, 7th Ed. (MacMillan Publishing Co. 1985). Other suitable toxins and/or chemotherapeutic agents are known to those of skill in the art.

In certain aspects, an OX40 agonist can be formulated so as to facilitate administration and promote stability of the active agent. In certain aspects, pharmaceutical compositions in accordance with the present disclosure include a pharmaceutically acceptable, non-toxic, sterile carrier such as physiological saline, non-toxic buffers, preservatives and the like. Suitable formulations for use in the treatment methods disclosed herein are described, e.g., in Remington's Pharmaceutical Sciences (Mack Publishing Co.) 16th ed. (1980).

### III. Interleukin-2 (IL-2), IL-2 Receptor, and Cytokines Binding to Common Gamma Chain Receptors

In certain aspects, methods of treating cancer are provided, where the methods include administration of an OX40 agonist with interleukin-2 or an active fragment, variant, analog, or derivative thereof. Interleukin-2 (IL-2) can, among other actions, enhance proliferation and activation of T cells and induce the secretion of a variety of cytokines (see, e.g., Bachmann, M F, and Oxenius, A. EMBO Rep 8:1142-1148 (2007)). IL-2 therapy (aldesleukin) has been approved by FDA for the treatment of metastatic renal cell carcinoma and metastatic melanoma. See, e.g., Jeal W Goa K L. BioDrugs. 1997 April; 7(4):285-317. Other IL-2-related drags in development include, but are not limited to BAY 50-4798, a high-affinity IL-2 analog which selectively targets T-lymphocytes over NK cells (Shanafelt A. et al., Nature Biotechnology 18, 1197-1202 (2000)), EMD 521873, an IL-2R-selective IL-2 mutant (see, e.g., Gillies S D, et al., Clin Cancer Res. 17:3673-85 (2011)), and IL-2/anti-IL-2 antibody complexes (see, e.g., Létourneau S, et al., Proc Natl Acad Sci USA. 107:2171-6 (2010)).

IL-2 binds to the trimeric IL-2 receptor (IL-2R), which includes IL-2Rα (CD25),/L-2/IL-15Rβ (CD122), and common gamma (yc; CD132) (Nelson B H, and Willerford D M. Adv Immunol 1998; 70: 1-81). Certain cells express a dimeric βγ receptor to which IL-2 binds with lower affinity but the same signal transduction capabilities (Krieg C. et al. Proc Natl. Acad Sci USA 107: 11906-11911 (2010)). In certain aspects, blocking the interaction of IL-2 with the CD25 portion of the receptor via CD122-directed IL-2/anti-IL-2 antibody complexes can block certain deleterious side effects of systemic IL-2 administration by lowering binding to the trimeric receptor present, e.g., on endothelial cells (Id.).

In certain aspects, methods of treating cancer are provided, where the methods include administration of an OX40 agonist, and a cytokine, or active fragment, variant, analog, or derivative thereof, that binds to a receptor with the common gamma chain. The common gamma chain (γc) (or CD132), also known as interleukin-2 receptor subunit gamma or IL-2RG, is a cytokine receptor sub-unit that is common to the receptor complexes for at least six different interleukin receptors: IL-2, IL-4, IL-7, IL-9, IL-15 and interleukin-21 receptor. As used herein, these cytokines which bind to receptors which include γc are referred to as “common gamma chain (γc) cytokines.” All of these cytokines utilize at least partially overlapping signal transduction pathways via JAK3-mediated phosphorylation of STAT3 and STAT5 (see, e.g., Kovanen P E, and Leonard W J. Immunol Rev 2004; 202: 67-83; Rochman Y, et al. Nat Rev Immunol 2009; 9: 480-90; Moroz A, et al. J Immunol 2004; 173: 900-9; and Sprent J, and Surh C D. Curr Opin Immunol 2001; 13: 248-54).

### IV. Methods for Treating Cancer

Provided herein are methods for treating cancer, where the methods include administration of an effective amount of an OX40 agonist and an effective amount common gamma chain (γc) cytokine or an active fragment, variant, analog, or derivative thereof, optionally in combination with other cancer treatments. Administration of an OX40 agonist results in an enhanced T-lymphocyte response to antigens on a variety of cancer cells, because the activation of OX40, while functioning in concert with antigenic stimulation of T-lymphocytes, is not antigen or cell-specific itself. Co-administration with a common gamma chain (γc) cytokine or an active fragment, variant, analog, or derivative thereof enhances OX40 expression.

In certain aspects, co-administration of an effective amount of an OX40 agonist and an effective amount common gamma chain (γc) cytokine or an active fragment, variant, analog, or derivative thereof stimulates T-lymphocyte-mediated anti-cancer immunity to a greater extent than the OX40 agonist or γc cytokine, e.g., IL-2, alone. Accordingly, an “effective amount” of either the OX40 agonist or the γc cytokine, IL-2, can, in some aspects, be less than the amount of each individual component administered independently. Similarly, co-administration, in some aspects, can allow for less frequent dosing. In certain aspects, the co-administration can restore the function of anergic tumor-reactive CD8⁺ T-lymphocytes, e.g., by restoring proliferation and/or differentiation of the anergic tumor-reactive CD8⁺ T-lymphocytes.

Also provided is a method of enhancing the effect of an OX40 agonist on T-lymphocyte-mediated cancer immunotherapy, where the method includes contacting T Cell Receptor (TCR)-stimulated T-lymphocytes with an OX40 agonist in combination with a γc cytokine, e.g., IL-2, or an active fragment, valiant, analog, or derivative thereof. Further provided is a method of enhancing the effect of an OX40 agonist on T-lymphocyte-mediated cancer immunotherapy, where the method includes stimulating T-lymphocytes via TCR ligation, and contacting the TCR-stimulated T-lymphocytes with an OX40 agonist in combination with a γc cytokine, e.g., IL-2, or an active fragment, variant, analog, or derivative thereof. Such methods can involve cancer immunotherapy requiring CD4⁺ T-lymphocytes, CD8⁺ T-lymphocytes, or both. In certain aspects, the T-lymphocyte-mediated cancer immunotherapy is enhanced to a greater extent than the OX40 agonist or γc cytokine, e.g., IL-2, alone. In certain aspects contacting TCR-stimulated T-lymphocytes with an OX40 agonist in combination with a γc cytokine, e.g., IL-2, or an active fragment, variant, analog, or derivative thereof can restore the function of anergic tumor-reactive T-lymphocytes, e.g., CD8⁺ T cells.

Also provided is a method of enhancing OX40 agonist-mediated augmentation of T-lymphocyte proliferation in response to TCR stimulation, where the method includes contacting TCR-stimulated T-lymphocytes with an OX40 agonist in combination with a γc cytokine, e.g., IL-2, or an active fragment, variant, analog, or derivative thereof. Further provided is a method of enhancing OX40 agonist-mediated augmentation of T-lymphocyte proliferation, where the method includes stimulating T-lymphocytes via TCR ligation, and contacting the TCR-stimulated T-lymphocytes with an OX40 agonist in combination with a γc cytokine, e.g., IL-2, or an active fragment, variant, analog, or derivative thereof. In certain aspects T-lymphocyte differentiation is also enhanced.

By “TCR ligation” is meant cross-linkage of TCR on the surface of T cells. In certain aspects, TCR ligation is accomplished through contacting T-lymphocytes with antigen/MHC complexes which specifically bind to the TCR. The antigen can be a cancer cell-specific antigen or an antigen which is preferentially expressed on cancer cells, e.g., a tumor antigen. In other aspects, TCR ligation is accomplished through contacting the T-lymphocytes with anti-CD3 which can be, e.g., bound to a solid substrate. Optionally the T-lymphocytes can also be contacted with anti-CD28. Suitable sources of anti-CD3 and anti-CD28 antibodies, e.g., monoclonal antibodies, e.g., both human and murine-CD3 and CD28-specific antibodies, are commercially available from sources well known to a person of ordinary skill in the art. In certain aspects TCR ligation according to this method is carried out in vivo, but can also be carried out in vitro or ex vivo.

In certain aspects of the treatment methods provided herein, the γc cytokine can be IL-2, IL4, IL7, IL-21, any active fragment, variant, analog or derivative thereof, and a combination thereof. In specific aspects, γc cytokine is IL-2 or an active fragment, variant, analog or derivative thereof, and a combination thereof. As described elsewhere herein, co-administration of an OX40 agonist with a γc cytokine, e.g., IL-2, can upregulate OX40 expression in the T-lymphocytes, thereby enhancing the immune-stimulating effects of OX40. While not wishing to be bound by theory, such upregulation can be mediated through JAK3 phosphorylation or other signal transduction pathways, which in turn can activate STAT5, STAT3, or both STAT5 and STAT3.

An effective amount of OX40 agonist and γc cytokine, e.g., IL-2, to be administered can be determined by a person of ordinary skill in the art by well-known methods. For example, in certain aspects an effective dose of an OX40 agonist, e.g., an anti-OX40 monoclonal antibody, is about 0.01 mg/kg to about 5.0 mg/kg, e.g., about 0.1 mg/kg, 0.4 mg/kg or 2 mg/kg of anti-OX40 mAb. Likewise, an effective does of a yc cytokine, e.g., IL-2, or fragment, variant, derivative, or analog thereof to be administered can be determined by a person of ordinary skill in the art by well-known methods. In certain aspects, the amount of γc cytokine, e.g., IL-2, to be administered is determined by balancing its synergistic effect on the OX40 agonist with the possibility of toxic side-effects. The OX40 agonist and γc cytokine, e.g., IL-2, can be administered as a single dose or as multiple doses, e.g., at least two, three, four, five, six or more doses, spaced at various time intervals to be determined by the attending physician, e.g., one or more doses a day, one or more doses every three days, one or more doses every five days, one or more doses every week, and so on. Treatment can continue or can be varied based on monitoring of efficacy (see below) for length of time to provide the most benefit to the patient being treated. Furthermore, the OX40 agonist and γc cytokine, e.g., IL-2, can be administered simultaneously, or one before the other, or alternating as multiple doses.

Clinical response to administration of an OX40 agonist and γc cytokine, e.g., IL-2 can be assessed, and optionally adjusted using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy with an OX40 agonist may experience the beneficial effect of an improvement in the symptoms associated with the disease.

Administration of the OX40 agonist and βc cytokine, e.g., IL-2, can be via any usable route, as determined by the nature of the formulation and the needs of the patient. In certain embodiments, the OX40 agonist is administered by IV infusion.

Given that immune stimulation with OX40 agonists is not antigen-specific, a variety of cancers can be treated by the methods provided herein, for example in certain aspects, the cancer is a solid tumor, or a metastasis thereof. Types of cancers include, but are not limited to melanoma, gastrointestinal cancer, renal cell carcinoma, prostate cancer, lung cancer, breast cancer, or any combination thereof. The site of metastasis is not limiting and can include, for example metastases in the lymph node, lung, liver, bone, or any combination thereof.

The cancer treatment methods provided herein can also include other conventional or non-conventional cancer treatments in addition to the administration of an OX40 agonist. By non-limiting example, administration of an OX40 agonist can be combined with surgery, radiation, chemotherapy, immunotherapy, targeting anti-cancer therapy, hormone therapy, or any combination thereof. The additional cancer therapy can be administered prior to, during, or subsequent to the administration of an OX40 agonist. Thus, where the combined therapies include administration of an OX40 agonist in combination with administration of another therapeutic agent, as with chemotherapy, radiation therapy, other anti-cancer antibody therapy, small molecule-based cancer therapy, or vaccine/immunotherapy-based cancer therapy, the methods described herein encompass coadministration, using separate formulations or a single pharmaceutical formulation, with simultaneous or consecutive administration in either order.

In certain methods of treating cancer as provided herein, the patient is a human patient. Effective treatment with an OX40 agonist in combination with a γc cytokine, e.g., IL-2, as described herein can include any favorable occurrence, e.g., reducing the rate of progression of the cancer, retardation or no increase in tumor or metastatic growth, stabilization of disease, prolonged survival of the patient, tumor shrinkage, or tumor regression, either at the site of a primary tumor, or in one or more metastases. In certain aspects of the methods of treating cancer as provided herein, effective treatment with an OX40 agonist in combination with a γc cytokine, e.g., IL-2, can retard, stall or decrease growth of a long-term established tumor or metastasis thereof.

The practice of embodiments encompassed by the disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevere, Amsterdam); Goldsby et al., eds. (2000) Kuby Immunology (4th ed.; H. Freemand & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlan); Sambrook and Russell (2001) Molecular Cloning: A Laboratory Manual (Cold Spring Harbor Press); Lewin (2003) Genes VIII (Prentice Ha112003); Harlow and Lane (1988) Antibodies: A Laboratory Manual (Cold Spring Harbor Press); Dieffenbach and Dveksler (2003) PCR Primer (Cold Spring Harbor Press).

All of the references cited above, as well as all references cited herein, are incorporated herein by reference in their entireties.

The following examples are offered by way of illustration and not by way of limitation.

### EXAMPLES

### General Methods

### Mice

Wild-type and CD25+/−C57BL/6 mice were purchased from Jackson Labs (Bar Harbor, Me.). OT-I Thy1.1 TCR Tg, (Prostate ovalbumin expressing transgenic) POET-1 Tg, OX40−/− OT-I TCR Tg, and STAT5a/b+/− mice were obtained from Dr. Charles Surh (The Scripps Research Institute, La Jolla, Calif.), Dr. Timothy Ratliff (Purdue University, West Lafayette, Ind.), Dr. Michael Croft (La Jolla Institute for Allergy and Immunology, La Jolla, Calif.), and Dr. Brad Nelson (BC Cancer Agency, Victoria, B C, Canada), respectively. C57BL/6 OX40-Cre mice were obtained from Dr. Nigel Killeen (UCSF, San Francisco, Calif.) and were crossed to mice carrying the Rosa26-loxP-STOP-loxP-YFP allele (Srinivas S, et al. BMC Dev Biol 2001; 1: 4). Splenocytes from STAT3−/− OT-I TCR Tg mice were obtained from Dr. Hua Yu (Beckman Research Institute at City of Hope, Duarte, Calif.). All mice were bred and maintained under specific pathogen-free conditions in the Providence Portland Medical Center animal facility. Experimental procedures were performed according to the National Institutes of Health Guide for the Care and Use of Laboratory Animals.

### Adoptive Transfer and Activation of OT-I T Cells In Vivo

Single cell suspensions were prepared from the lymph nodes and spleens of OT-I Thy1.1 TCR Tg mice. Cell suspensions were depleted of CD4⁺, CD11b⁺, CD45R⁺, DX5⁺, and Ter-119⁺ cells using the Dynal mouse CD8 cell negative isolation kit (Invitrogen, Carlsbad, Calif.). OT-I T cells were purified by negative selection per the manufacturer's instructions and had a naïve phenotype (CD25-negative, CD44^{low}, CD62L^{hi}, and CD69-negative) as indicated by flow cytometry (data not shown). Donor OT-I T cells were injected i.v. in 200 μl of PBS into recipient mice.

Where indicated, recipient mice received 500 μg of soluble ovalbumin (Sigma, St. Louis, Mo.), 50 μg of anti-OX40 (clone OX86) or control rat IgG Ab (Sigma), and/or 10 μg bacterial lipopolysaccharide (LPS) (Sigma) s.c. Mice received an additional dose (50 μg) of anti-OX40 or control Ab one day later. For cell depletion, tumor-bearing mice were treated with 200 μg (i.p.) anti-CD4 (clone GK1.5; Bio X Cell, West Lebanon, N.H.) and/or anti-CD8 (clone 53-6.72; Bio X Cell) at the indicated time points.

### Lymphocyte Isolation and Analysis

Lymph nodes were harvested and processed to obtain single cell suspensions. ACK lysing buffer (Lonza, Walkersville, Md.) was added for 5 min at RT to lyse red blood cells. Cells were then rinsed with RPMI 1640 medium (Lonza) containing 10% FBS (10% cRPMI) (Lonza) supplemented with 1M HEPES, non-essential amino acids, sodium pyruvate (all from Lonza), and pen-strep glutamine (Invitrogen).

Murine peripheral blood lymphocytes were collected via the tail vein into tubes containing 50 μl heparin (Hospira, Lake Forest, Ill.). One ml of flow cytometry wash buffer (0.5% FBS, 0.5 mM EDTA, and 0.02% NaN3 in PBS) was added, cells were mixed, and then 700 μl of Ficoll-Paque (GE Healthcare, Piscataway, N.J.) was added prior to centrifugation. Lymphocytes were collected from the interface and then washed with flow cytometry buffer prior to staining. Cells were incubated for 30 min at 4° C. with: Ki-67 FITC, Thy1.1 PE-Cy7, Thy1.1 eFluor 450, OX40 PE, granzyme B PE, CD3 eFluor 710, CD8 eFluor 605, CD8 PE-Cy7, KLRG-1 APC, CD25 eFluor 488, CD25 Alexa Fluor 700, Fixable Viability Dye eFluor 780, or CD4 V500. Human cells were incubated with CD3 APC-H7, CD4 PerCP-Cy5.5, CD8 PE-Cy7, APC CD25 and OX40 PE. All antibodies were obtained from eBioscience (San Diego, Calif.), BD Biosciences (San Jose, Calif.), BioLegend (San Diego, Calif.), Miltenyi Biotec (Bergisch Gladbach, Germany), or Invitrogen. For intracellular staining, cells were fixed and permeabilized with the Foxp3 Staining Buffer Set (eBioscience) according to the manufacturer's instructions. Cells were analyzed with an LSR II flow cytometer using FACSDiva software (BD Biosciences).

### Isolation and Stimulation of Human PBMC

Human PBMC from healthy donors were isolated by centrifugation of heparinized blood over Ficoll-Paque PLUS (GE Healthcare). The Providence Health System Institutional Review Board approved the study and all blood donors gave their informed consent. Fresh human PBMC were enriched for CD4⁺ and CD8⁺ T cells by negative selection using a CD4 or CD8 T cell negative isolation kit (Miltenyi Biotec) and suspended in 10% cRPMI (5×10⁵ cells/ml) and stimulated with 1 μg/ml plate bound anti-CD3 (clone OKT-3) in 96-well plates with or without 5,000 U/ml of rhIL-2 (Proleukin). After 48 hours, cells were washed, re-suspended, and then plated in 96-well plates with or without 5,000 IU/ml of rhIL-2. Cells were stained and analyzed by flow cytometry 24 hours later.

### T Cell Activation In Vitro

Single cell suspensions were prepared from the lymph nodes and spleens of wild-type, CD25−/−, STAT3−/−, or STAT5−/− mice and then CD4⁺ or CD8⁺ T cells were purified using the Dynal mouse CD4 or CD8 T cell negative isolation kit (Invitrogen). 3×10⁵ cells per well were seeded into 96-well plates containing plate-bound anti-CD3 (1 μg/ml; clone 145-2C11) and anti-CD28 (5 μg/ml; clone 37.51). For antigen-specific CD8⁺ T cell activation, purified naïve wild-type or OX40−/− OT-I T cells (2×10⁵/well) were stimulated with OVA peptide (SIINFEKL)-pulsed irradiated (20,000 rads) DC2.4 cells (2×10³/well) in 96-well plates. Alternatively, purified naïve wild-type OT-I, STAT3−/− OT-I, or OX40−/− OT-I T cells (1×10⁶/well) were stimulated with wild-type cognate (SIINFEKL) or altered peptide ligand (SIITFEKL) OVA peptide-pulsed irradiated (2,000 rads) syngeneic splenocytes (6×10⁶/well) in 24-well plates. Forty-eight hours later, activated OT-I T cells were harvested and live cells were enriched over a Ficoll-paque gradient prior to re-seeding in fresh 10% cRPMI (5×10⁵ cells/ml).

### Treg Functional Assay.

MCA-205 tumors were implanted into wild-type C57BL/6 mice and then 10 days later, mice received 250 μg anti-OX40 or control rat Ig (d10, 14) in the presence or absence of IL-2c (d10-13). Seven days later (d21 post-tumor implantation), spleens were harvested, RBC lysed, and CD4⁺ CD25⁺ regulatory T cells (CD8⁻/MHC II⁻/B220⁻) were isolated by cell sorting (>99% purity). Treg were seeded in triplicate at 5×10⁴ cells/well in 96-well round-bottom plates. Naïve responder (Teff) CD8 cells were prepared from the spleens of wild-type mice using the Dynal CD8 T cell negative selection kit (Invitrogen), CFSE-labeled, and 5×10⁴ cells/well were added to triplicate wells containing media (positive control) or Treg cells. 2×10⁵ irradiated (4,000 rads) T-cell depleted (Dynal beads, Invitrogen) accessory cells were prepared, treated with 1 μg/ml anti-CD3 and added to all wells. Cells were harvested 96 hours later, stained for CD8, and the extent of CFSE dilution in the CD8 responder cells was determined by flow cytometry.

### Cytokines and Inhibitors

Recombinant murine IL-2, IL-4, IL-7, IL-9, or IL-21 were purchased from eBioscience or Peprotech (Rocky Hill, N.J.). Recombinant human IL-15 was provided by the National Cancer Institute's Biological Resources Branch and anti-mIL-2 mAb (clone S4B6) was obtained from Bio-X-Cell. IL-2/anti-IL-2 mAb complexes (IL-2c) were generated by mixing 2.5 μg IL-2 with 7 μg anti-IL-2 mAb for 20 min at 37° C. and then mice received daily injections of IL-2c in 200 μl PBS (i.p.). Where indicated, T cells were treated in vitro with a JAK3 inhibitor (100 ng/ml; PF-956980; obtained from Pfizer).

### Tumor Challenge and Anergy Induction

1×10⁶ MCA-205 sarcoma tumor cells were implanted into wild-type C57BL/6 mice (s.c.) (Spiess P J, et al. J Natl Cancer Inst 1987; 79: 1067-75). TRAMP-C1-mOVA (TC1-OVA) cells were modified to express membrane-bound OVA (mOVA) as previously described (Redmond W L, et al. J Immunol 2007; 179: 7244-53). In some experiments, 2.5×10⁶ TC1-OVA cells were injected into male POET Tg mice (s.c.). When tumors reached ˜50 mm² (20 days post-tumor inoculation), mice received either 5×10⁵ wild-type or OX40−/− OT-I Thy1.1 T cells. Seventeen days after CD8 T cell adoptive transfer, anergic donor cells in tumor-bearing mice were re-challenged with soluble OVA, anti-OX40 or control Ab, and LPS (s.c.) as described above. Tumor growth (area) was assessed every 2-3 days with micro-calipers and mice were sacrificed when tumors reached >150 mm².

### In Vivo Cytolytic Assay

Target cells, comprised of syngeneic splenocytes, were labeled with 5 μM carboxyfluorescein diacetate succinimidyl ester (CFSE) (CFSE^{high}) or 0.5 μM CFSE (CFSE^{low}) in 1×PBS for 10 minutes at 37° C. and then washed twice with 10% cRPMI. Next, CFSE^{low} and CFSE^{high} cells were pulsed with 5 μg/ml control (HA) or cognate (OVA) peptide, respectively, for 1 h at 37° C. Cells were washed twice with 10% cRPMI and then a 1:1 mixture of CFSE^{low}/CFSE^{high} target cells (5×10⁶/each) were injected i.v. in 1X PBS into recipient mice. Four hours later, splenocytes were harvested and single cell suspensions were analyzed for detection and quantification of CFSE-labeled cells by flow cytometry.

### Western Blotting

Whole cell lysates were prepared using RIPA lysis buffer (Bio-Rad, Hercules, Calif.) containing HALT protease inhibitor cocktail (Thermo Fisher Scientific, Rockford, Ill.) for 30 min at 4° C. Lysates were centrifuged at 14,000×g/4° C., supernatants were collected, protein concentration was determined by Bradford assay kit (ISC BioExpress, Kaysville, Utah) and 50 μg aliquots were stored at −80° C. Lysates were boiled at 100° C. for 5 min in Laemmli buffer (Invitrogen) containing 2-ME, resolved by SDS-PAGE on 12% pre-cast gels (Bio-Rad), and then transferred to nitrocellulose membranes (Invitrogen). Non-specific binding was reduced by blocking with a 1:1 mixture of Odyssey Blocking buffer (Li-Cor, Lincoln, Nebr.) and 1X PBS or 5% non-fat dry milk in 1X PBS for 1 hour at RT. Blots were incubated with Abs against pJAK1, pJAK2, pSTAT1, pSTAT3, pSTAT5, pSTAT6, JAK1, JAK2, STAT1, STAT3, STAT4, STAT5, STAT6 (all from Cell Signaling, Danvers, Mass.), pJAK3, JAK3 (Santa Cruz Biotechnology, Santa Cruz, Calif.), pSTAT4 (Invitrogen), GAPDH (Sigma), or beta-actin (Li-Cor) in Odyssey (Li-Cor) blocking buffer overnight at 4° C. Blots were washed 4×5 min at RT with PBS-Tween (1×PBS+0.2% Tween-20) and then incubated with IRDye 800CW goat anti-rabbit IgG (H+L), IRDye 680LT goat anti-mouse IgG (H+L), or IRDye 680LT donkey anti-Goat IgG (H+L) (Li-Cor) for 60 min at RT. Blots were washed 4×5 min at RT with PBS-Tween and then rinsed briefly with 1×PBS prior to visualization on a Li-Cor Odyssey infrared imager (Li-Cor).

### Statistical Analysis

Statistical significance was determined by unpaired Student's t-test (for comparison between 2 groups), one-way ANOVA (for comparison among >2 groups), or Kaplan-Meier survival (for tumor survival studies) using GraphPad InStat or Prism software (GraphPad, San Diego, Calif.); a P value of <0.05 was considered significant.

### Example 1

### Optimal OX40 Expression is Regulated by the Strength of TCR Stimulation and IL-2Rα (CD25)

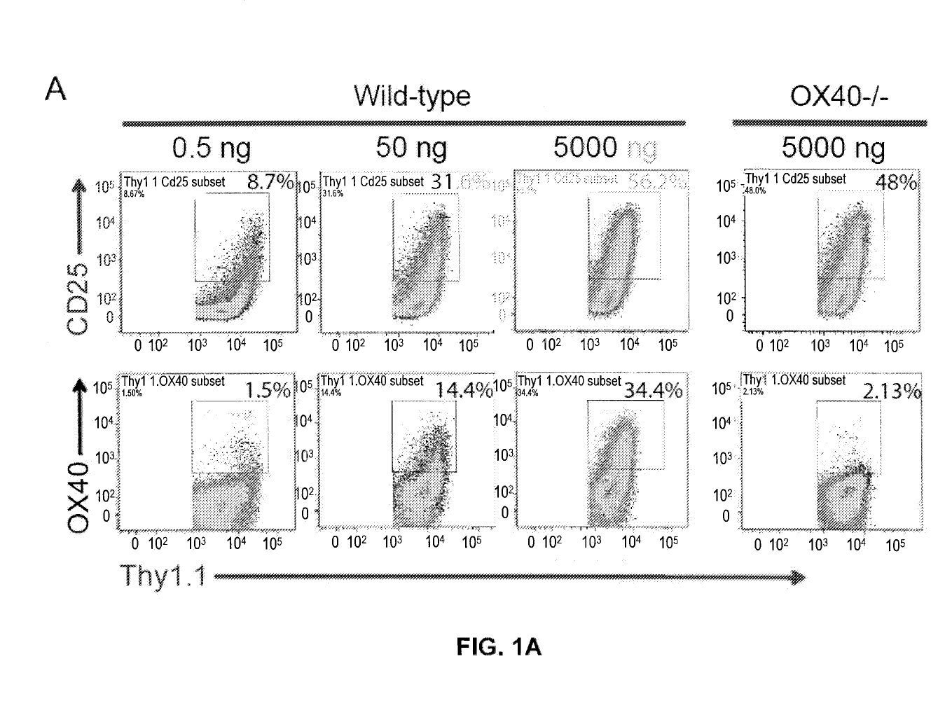

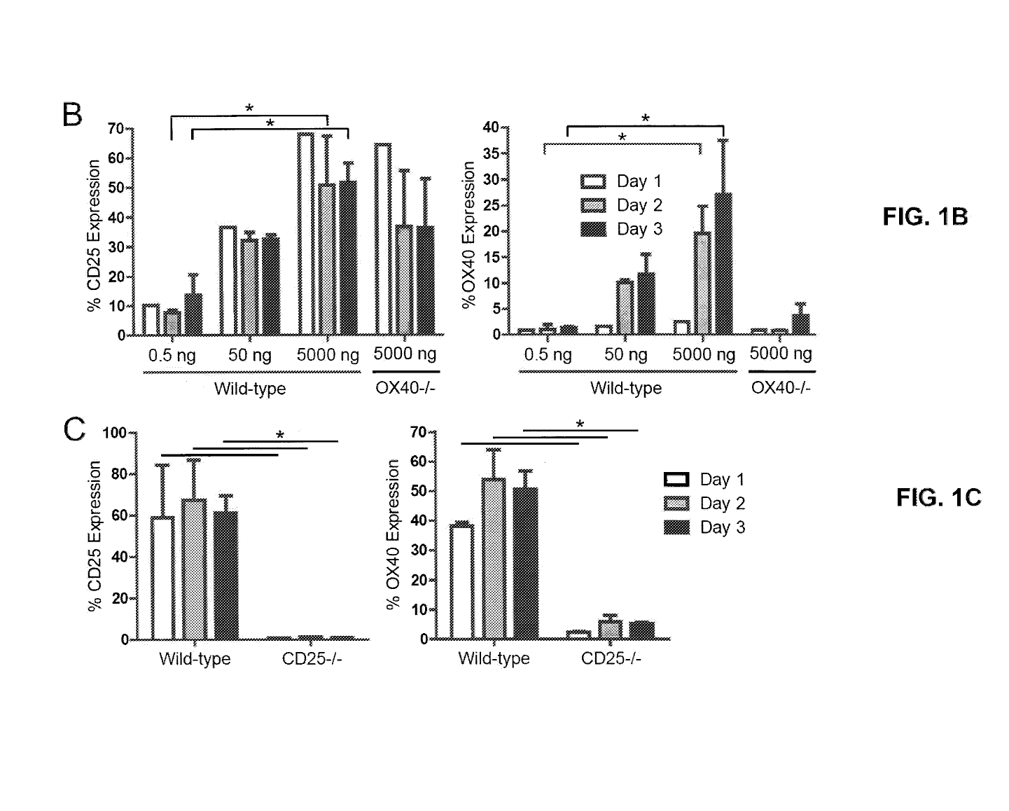

The extent to which the strength of TCR stimulation affects OX40 expression, the kinetics of OX40 up-regulation following the activation of naïve CD8 T cells was assessed as follows. Purified naïve wild-type or OX40−/− OT-I T cells (2×10⁵/ml) were activated with syngeneic antigen presenting cells (APCs) (2×10³/ml) pulsed with increasing doses (0.5 ng, 50 ng, or 5000 ng) of the OVA peptide, SIINFEKL. One to three days later, activated OT-I T cells were harvested and the expression of OX40 and CD25 was determined by flow cytometry. CD25 was rapidly up-regulated and reached maximal expression within 24 hrs after TCR stimulation at the highest dose of Ag (5000 ng/ml) whether or not OX40 was expressed (FIG. 1A, 1B). Maximal OX40 expression was similarly induced in a dose-dependent manner with peak OX40 expression observed 3 days post-stimulation in the OX40-expressing cells (FIG. 1A, 1B). The bar graphs in FIGS. 1B and 1C depict the mean+/− SD (n=2-3/group). Data are representative of one out of two to three independent experiments with similar results.

The effects of IL-2 on OX40 expression on T cells was then determined. Purified naïve polyclonal wild-type or CD25−/− CD8 T cells (3×10⁵/well) were CFSE-labeled and then stimulated with plate-bound anti-CD3 and anti-CD28 (1 and 5 μg/ml, respectively). One to three days later, the activated CD8 T cells were harvested and the extent of CD25 and OX40 expression was determined by flow cytometry. CD25 and OX40 were both induced on wild-type T cells (FIG. 1C), while CD25−/− CD8 T cells expressed little or no OX40 following TCR stimulation (FIG. 1C), demonstrating that TCR stimulation alone was not sufficient to drive robust expression of OX40. Similar results were obtained following stimulation of murine polyclonal CD25−/− CD4⁺ T cells (data not shown), demonstrating that expression of the high-affinity IL-2R complex is required for optimal induction of OX40 on T cells.

This example demonstrates that the initial expression of OX40 is regulated in part through the strength of TCR engagement as strong TCR ligation with high doses of antigen induced higher levels of OX40 expression than low doses of antigen (FIG. 1). Although TCR stimulation was necessary to induce OX40, TCR ligation alone was not sufficient to drive robust expression of OX40. A role for IL-2/IL-2R signaling in regulating OX40 expression was found. In particular, IL-2Rα-deficient T cells exhibited a marked defect in their ability to up-regulate OX40 following TCR ligation (FIG. 1C).

### Example 2

### Exogenous IL-2 Up-Regulates OX40 on Activated Murine and Human T Cells

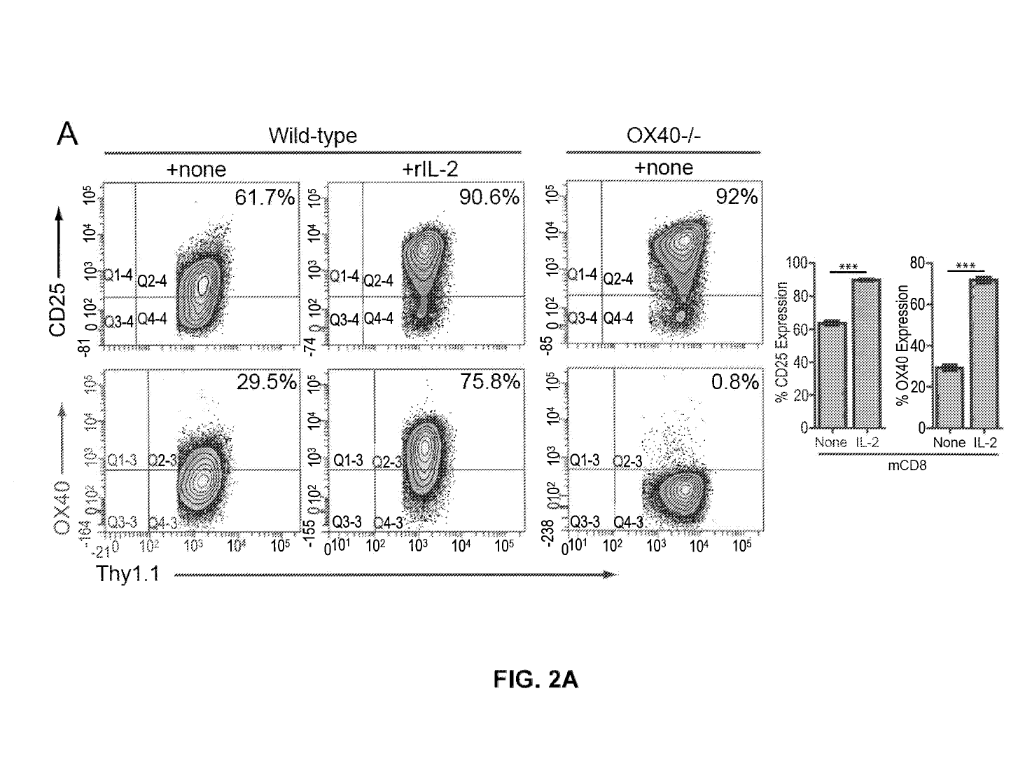

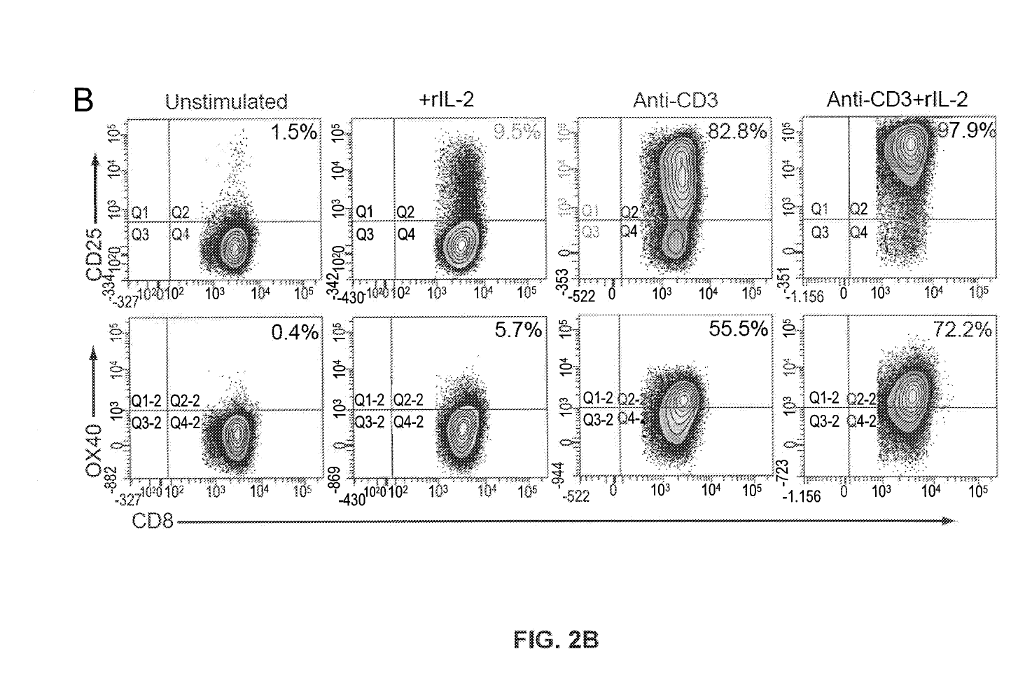

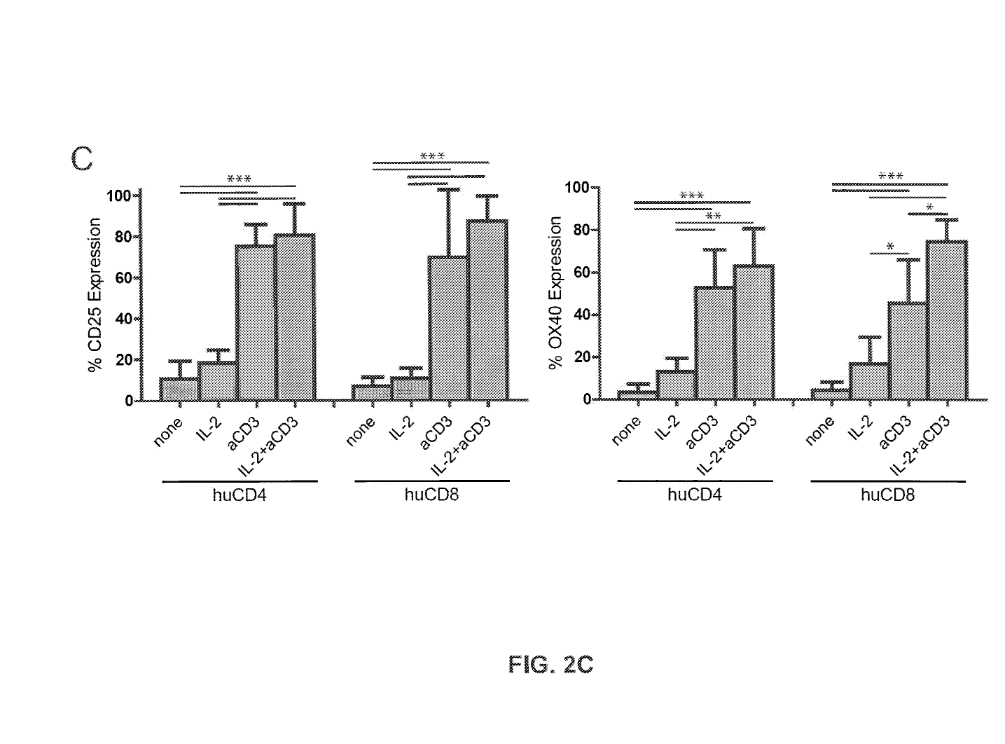

Whether the addition of exogenous rIL-2 was sufficient to up-regulate OX40 on activated T cells was determined as follows. Purified naïve wild-type or OX40−/− OT-I T cells (1×10⁶/ml) were activated with cognate peptide-pulsed syngeneic splenocytes (6×10⁶/ml). Two days later, activated OT-I T cells were harvested and re-cultured (5×10⁵ cells/nil) in the presence or absence of recombinant murine IL-2 (100 ng/ml). The extent of CD25 and OX40 expression was determined by flow cytometry. The addition of exogenous rIL-2 led to a statistically significant increase in both CD25 and OX40 expression compared to media alone (FIG. 2A), demonstrating that IL-2 signaling was sufficient to drive up-regulation of these molecules on activated murine T cells.

Whether TCR stimulation plus exogenous rIL-2 similarly regulated OX40 expression on human T cells was examined as follows. Purified human CD8⁺ or CD4⁺ T cells were collected from PBMC and were stimulated with media, plate-bound recombinant human IL-2 (5,000 IU/ml, equivalent to 300 ng/ml), and/or plate-bound 1 μg/ml anti-CD3 mAb (OKT-3). Forty-eight hours later, the stimulated cells were harvested, washed, and then stimulated with media or recombinant human IL-2 (5,000 IU/ml). Twenty-four hours later, the extent of CD25 and OX40 expression were measured by flow cytometry. Neither CD25 nor OX40 expression was detected on un-stimulated CD8⁺ and CD4⁺ T cells, but they were both modestly induced following exposure to IL-2 (FIGS. 2B, 2C). Although stimulation with anti-CD3 alone led to significantly increased CD25 expression, combined IL-2 and TCR stimulation trended towards increased OX40 expression on human CD4⁺ T cells (FIG. 2C) and a statistically significant increase in OX40 on human CD8⁺ T cells (FIGS. 2B, 2C). Together, these data demonstrate that the combination of TCR/IL-2R stimulation can induce optimal expression of OX40 on murine and human T cells.

This example demonstrates that TCR ligation in the presence of exogenous IL-2 was sufficient to promote robust expression of OX40 on both murine and human CD4⁺ and CD8⁺ T cells (FIG. 2). While not being bound by theory, this result suggests that IL-2-mediated enhancement of OX40 expression is part of a conserved mechanism of regulating OX40.

### Example 3

### OX40 Expression is Regulated by JAK3, STAT5, and STAT5

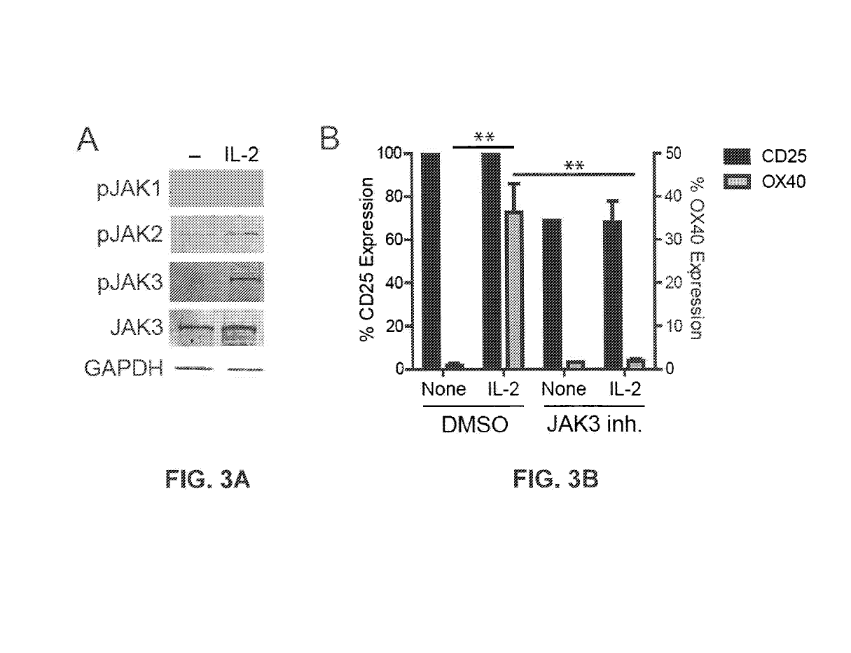

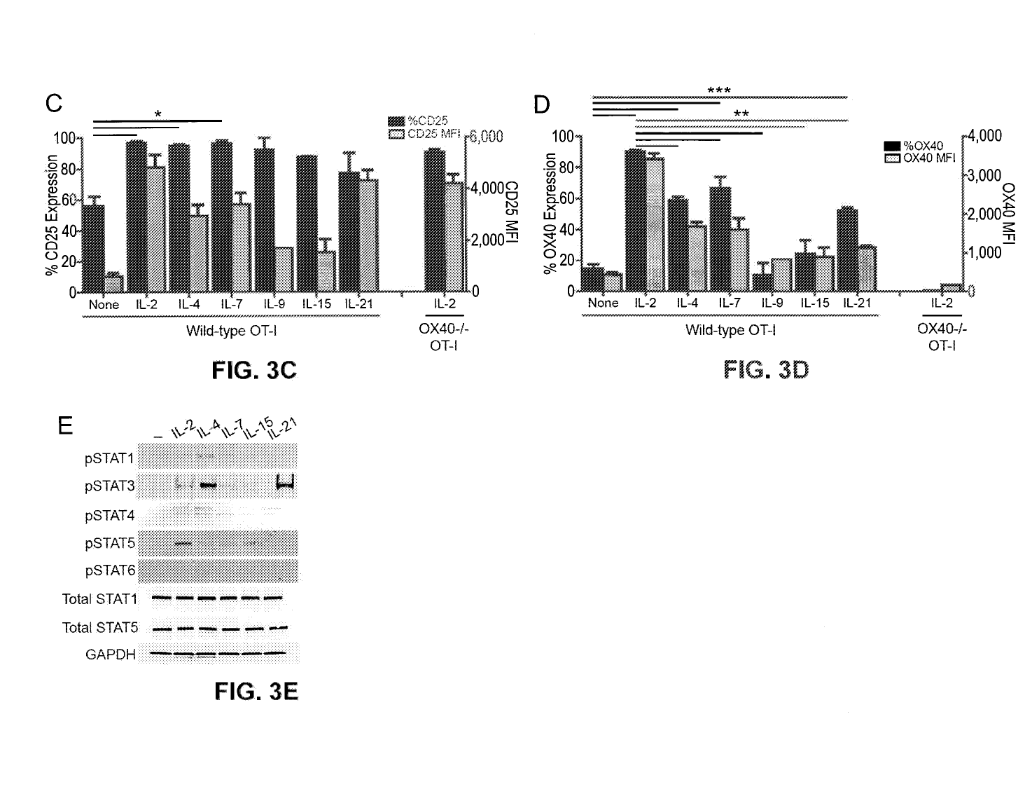

The tyrosine kinase JAK3 binds to the common γc subunit and its phosphorylation is a critical factor in the proximal downstream signaling following stimulation with γc cytokines (Kovanen P E, and Leonard W J. Immunol Rev 2004; 202: 67-83; Rochman Y, et al. Nat Rev Immunol 2009; 9: 480-90). Whether JAK3 activation is required to induce OX40 expression was examined as follows. First, the expression of JAK proteins in CD8⁺ T cells stimulated in vitro was assessed. Antigen-specific CD8⁺ T cells from OT-1 transgenic mice (as in Examples 1 and 2) were used for these studies in order to control more precisely the extent and duration of TCR stimulation. Naïve wild-type or OX40−/− OT-I T cells were activated for two days with peptide-pulsed APCs as described above. The activated OT-I T cells were then harvested and re-cultured (5×10⁵ cells/ml) with media or recombinant murine IL-2 (100 ng/ml), and the expression of phosphorylated JAK1, JAK2, and JAK 3, as well as total JAK3 was assessed by Western blot. Stimulation with rIL-2 led to increased phosphorylation of JAK3, but did not affect JAK1 or JAK2 phosphorylation, suggesting that JAK3 signaling is responsible for the up-regulation of OX40 (FIG. 3A). The requirement for JAK3 was confirmed by culturing activated CD8⁺ T cells with media or IL-2 in the presence or absence of a JAK3-specific small molecule inhibitor (PF-956980, 100 ng/ml) (Changelian P S, et al. Blood 2008; 111: 2155-7; Steele A J, et al. Blood 2010). Twenty-four hours later, cells were harvested and the extent of CD25 and OX40 expression was determined by flow cytometry. Treatment with the JAK3 inhibitor abrogated the IL-2-mediated induction of OX40 on activated CD8⁺ T cells compared to control-treated cells (DMSO) (FIG. 3B).

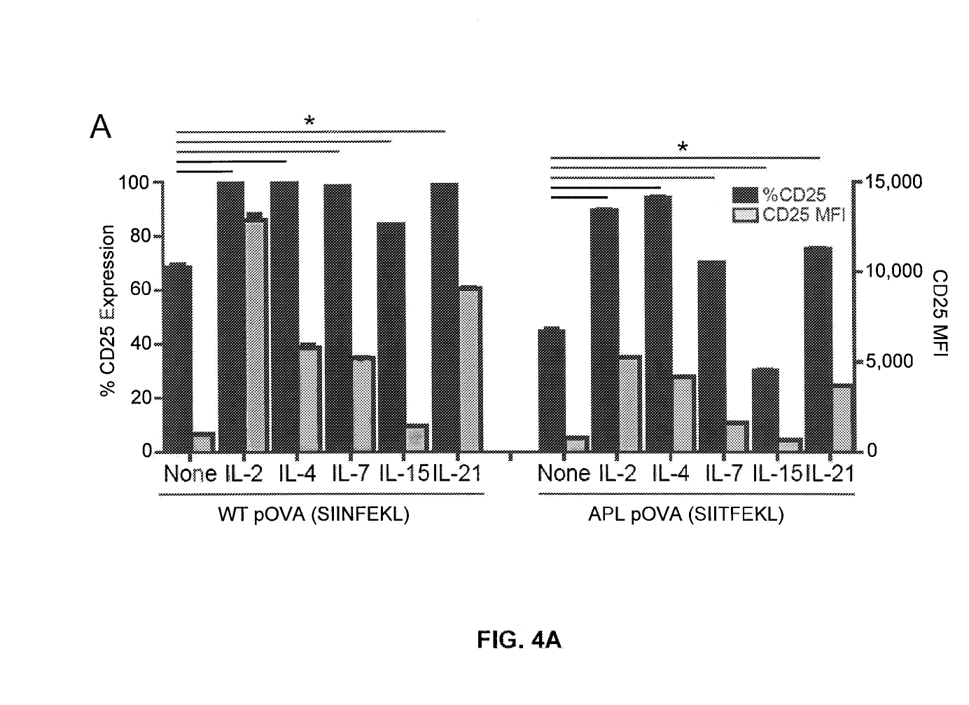

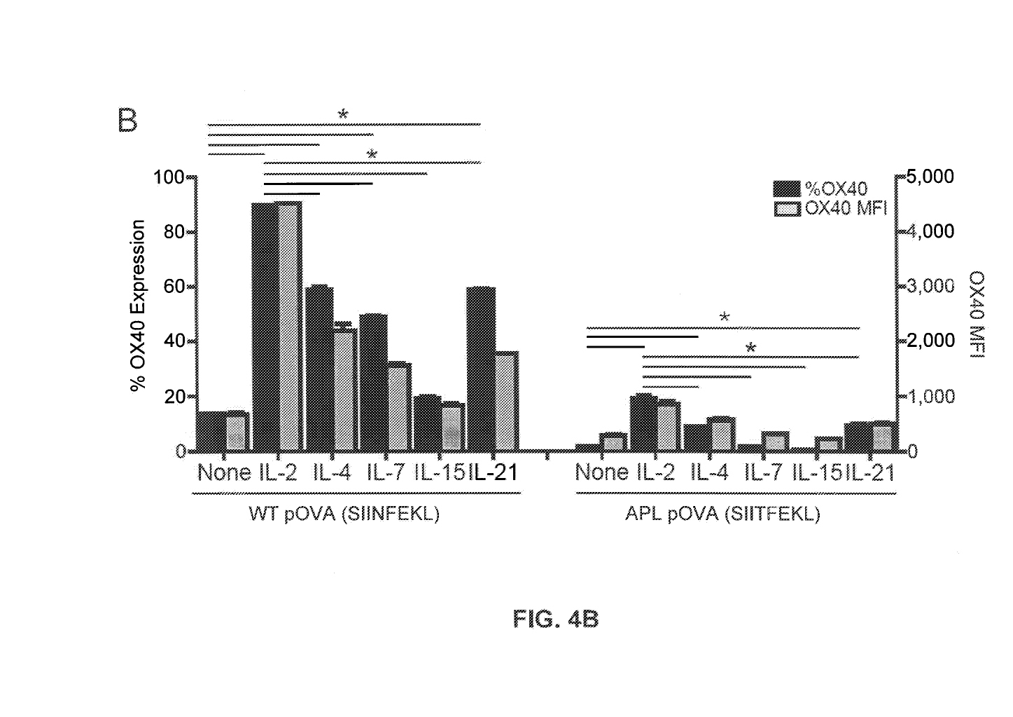

The γc subunit is constitutively expressed and shared among the following cytokines: IL-2, IL-4, IL-7, IL-9, IL-15, and IL-21. Despite sharing the common yc subunit, the majority of IL-2 family cytokines signal through a complex consisting of a unique alpha chain paired with the shared yc, which leads to distinct downstream effects on T cell survival and differentiation (Nelson B H, and Willerford D M. Adv Immunol 1998; 70: 1-81; Gaffen S L. Cytokine 2001; 14: 63-77; Kovanen P E, and Leonard W J. Immunol Rev 2004; 202: 67-83). To determine how the different γc cytokines affected OX40 expression, WT or OX40−/− OT-I cells were activated for two days with peptide-pulsed APCs as described above, harvested, and then stimulated with media alone, recombinant murine IL-2, recombinant murine IL-4, recombinant murine IL-7, recombinant murine IL-9, recombinant murine IL-15, or recombinant murine IL-21 (100 ng/ml) OT-I T cells were cultured in the presence of recombinant murine IL-2, IL-4, IL-7, IL-9, IL-15, or IL-21. Twenty-four hours later, cells were harvested and the extent of CD25 and OX40 expression was determined by flow cytometry. While all the yc cytokines tested were able to induce increased expression of CD25 (FIG. 3C), IL-2 stimulation promoted the greatest increase in OX40 expression (FIG. 3D, % OX40⁺). Stimulation with IL-4, IL-7, or IL-21 led to a modest up-regulation of OX40 (FIG. 3D; % OX40⁺), while IL-9 and IL-15 did not affect OX40 expression (FIG. 3D). The ability of yc cytokines to induce OX40 was also tested following CD8 T cell activation with a low-affinity altered peptide ligand (SIITFEKL), which exhibits ˜700-1,000-fold decrease in TCR affinity as compared to the native SIINFEKL epitope (Zehn D, et al. Nature 2009; 458: 211-4). Purified naïve OT-I T cells were stimulated with wild-type (SIINFEKL) or altered peptide ligand (SIITFEKL) pOVA-pulsed APCs by the methods described previously. Two days later, the activated OT-I T cells were harvested, re-cultured (5×10⁵ cells/ml), and then stimulated with media alone, or with recombinant murine IL-2, IL-4, IL-7, IL-15, or IL-21 (100 ng/ml). Twenty-four hours later, cells were harvested and the extent of CD25 OX40 expression (% positive and MFI) were determined by flow cytometry. Although the extent of maximal OX40 expression was reduced following stimulation with low-affinity OVA peptide (˜20% vs. 90% with WT pOVA; FIG. 4B), the hierarchy of CD25 and OX40 induction (IL-2>>>IL-4, IL-7, IL-21) was maintained (FIGS. 4A and 4B, respectively).

Stimulation with γc cytokines and JAK3 promotes T cell activation and survival through three major pathways, PI3K/AKT, MAPK/ERK, and the activation of STAT transcription factors (Leonard W J, and O'Shea J J. Annu Rev Immunol 1998; 16: 293-322). The pathway responsible for regulating OX40 was determined as follows. First, no change in the IL-2-mediated induction of OX40 expression was observed following CD8 T cell activation in the presence of PI3K or AKT inhibitors (data not shown). Similarly, wild-type and ERK2−/− CD8⁺ T cells expressed similar amounts of OX40 (data not shown), demonstrating that OX40 was induced independently of PI3K/AKT or ERK. The role of STAT signaling in driving OX40 expression was then investigated. WT OT-I T cells were activated for two days with peptide-pulsed APCs as described above, and were then re-stimulated with media alone, or with the common γc cytokines IL-2, IL-4, IL-7, IL-15 and IL-21. As seen in FIG. 3E, IL-2 stimulation led to a robust increase in STAT5 phosphorylation, while IL-4, IL-7, and IL-15 caused lower levels of STAT5 phosphorylation (FIG. 3E). IL-21 and IL-4 induced high levels of STAT3 phosphorylation, while IL-2 weakly induced STAT3 phosphorylation. Further analysis revealed no differential expression and only low levels of STAT1, STAT4, and STATE phosphorylation (FIG. 3E).

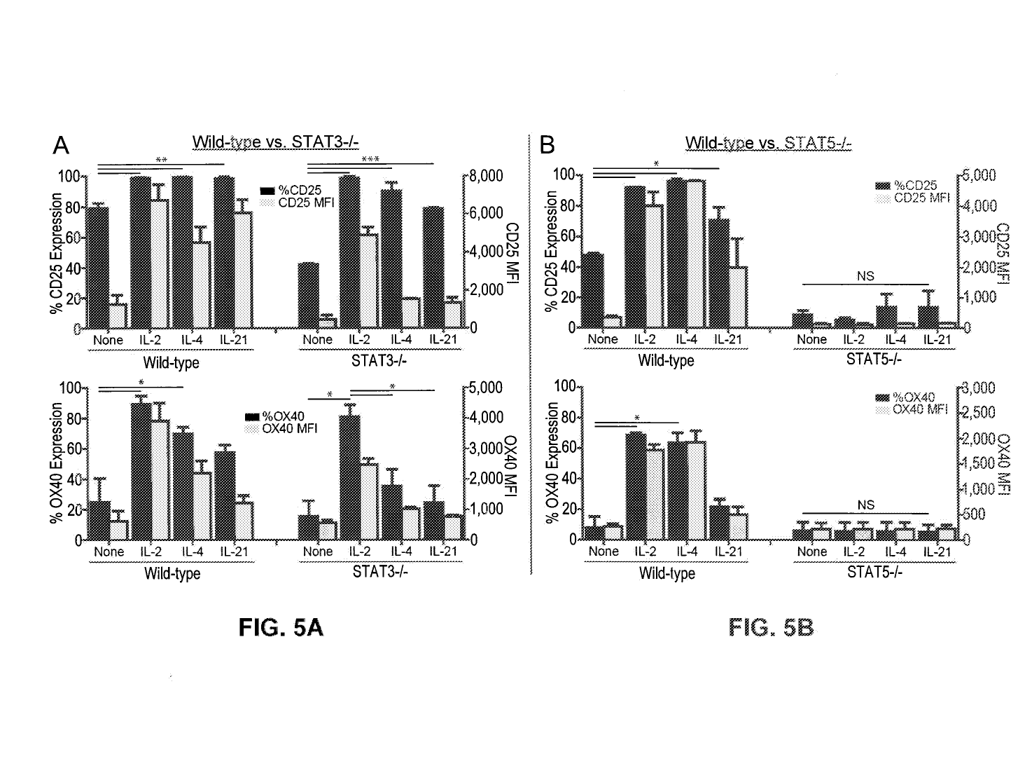

The contribution of STAT3 and STAT5 to the regulation of OX40, wild-type, STAT3−/−, or STAT5−/− CD8⁺ T cells was tested as follows. First, WT or STAT3−/− OT-I T cells were activated for two days with peptide-pulsed APCs as described above and then stimulated with media alone, or with recombinant murine IL-2, IL-4, or IL-21 (100 ng/ml), the cytokines which had previously been shown to up-regulate CD25 and OX40, and induce strong phosphorylation of STAT3 and/or STAT5. 24 hours later cells were harvested and the extent of CD25 and OX40 expression (% positive and MFI) was measured by flow cytometry. The results are shown in FIG. 5A. Then, polyclonal endogenous WT or STAT5−/− CD8⁺ T cells were stimulated for 2 days with 2 μg/ml anti-CD3 mAb, harvested, and then re-cultured and stimulated with media alone, or with recombinant murine IL-2, IL-4, or IL-21 (100 ng/ml). The cells were harvested 24 hours later, and the extent of CD25 and OX40 expression (% positive and MFI) was determined by flow cytometry. The results are shown in FIG. 5B. Both wild-type and STAT3−/− CD8⁺ T cells up-regulated CD25 following stimulation with γc cytokines, although STAT3−/− CD8⁺ T cells exhibited reduced expression (% positive and MFI) compared to wild-type cells, particularly following stimulation with IL-4 or IL-21 (FIG. 5A). However, only IL-2 induced statistically significant up-regulation of OX40 on STAT3−/− CD8⁺ T cells (FIG. 5A; OX40⁺).

STAT5-deficient CD8⁺ T cells were unable to induce CD25 or OX40 expression following stimulation with IL-2, IL-4, or IL-21, indicating an essential role for STAT5 in driving γc cytokine-mediated up-regulation of CD25 and OX40 (FIG. 5B). Similar results were obtained using either TCR transgenic OT-I T cells (FIG. 5A) or endogenous polyclonal CD8⁺ T cells (FIG. 5B and data not shown). Together, these studies demonstrated that γc cytokines regulate OX40 via unique mechanisms as IL-2 drove OX40 expression in a primarily STAT3-independent and STAT5-dependent manner, while IL-4 and IL-21 induced OX40 via a dual STAT3/STAT5-dependent mechanism.

Mechanistic studies revealed that IL-2 stimulation induced JAK3 phosphorylation, which in turn was required for optimal induction of OX40 (FIG. 3A, 3B). Additional investigation demonstrated a hierarchy in which IL-2 consistently drove the most robust expression of OX40, while IL-4, IL-7, and IL-21 were less efficient at inducing OX40 (FIG. 3D). In contrast, IL-9 and IL-15 did not up-regulate OX40 (FIG. 3D). It should be noted that a similar hierarchy of γc cytokine-mediated induction of OX40 was obtained following stimulation of TCR Tg OT-I T cells or endogenous polyclonal CD8⁺ T cells with wild-type pOVA (FIG. 3), a low-affinity altered peptide ligand pOVA (FIG. 4), or anti-CD3 (FIG. 5B). The molecular basis for the discordant effects of IL-15 versus IL-2/IL-4/IL-7/IL-21 stimulation remain unclear since all of these cytokines utilize at least partially overlapping signal transduction pathways via JAK3-mediated phosphorylation of STAT3 and STAT5 (FIG. 3E, and see, e.g., Kovanen P E, and Leonard W J. Immunol Rev 2004; 202: 67-83; Rochman Y, et al. Nat Rev Immunol 2009; 9: 480-90; Moroz A, et al. J Immunol 2004; 173: 900-9; Sprent J, and Surh C D. Curr Opin Immunol 2001; 13: 248-54). While not wishing to be bound by theory, some possibilities include the regulation by adapter proteins like Gab2, negative regulators of STATs such as SOCS proteins, epigenetic changes, as well as differential activation and/or binding of STAT5a versus STAT5β isoforms to the OX40 promoter (see, e.g., Gadina M, et al. J Biol Chem 2000; 275: 26959-66; Basham B, et al. Nucleic Acids Res 2008; 36: 3802-18; and Teglund S, et al. Cell 1998; 93: 841-50).

In order to determine whether differences in the homo-versus hetero-dimerization of STAT3 and STAT5 or in the binding of dimeric versus tetrameric STAT5 proteins to the OX40 promoter could account for differences in STAT3 versus STAT5-dependent induction of OX40 (FIG. 5), the putative STAT3 and STAT5-binding sites in the OX40 promoter have been determined (data not shown) in order to elucidate the transcriptional machinery regulating OX40 expression.

### Example 4

### Combined Anti-OX40 mAb/IL-2 Therapy Boosts Anti-Tumor Immunity

Numerous pre-clinical studies have demonstrated that treatment with an agonist anti-OX40 mAb promotes potent anti-tumor immunity (Watts T H, Annu Rev Immunol 2005; 23: 23-68; Redmond W L and Weinberg A D, Crit Rev Immunol 2007; 27: 415-36; Croft M. Annu Rev Immunol 2010; 28: 57-78). Based upon the ability of exogenous IL-2 to strongly induce OX40 in vitro (FIG. 2), whether the provision of IL-2 therapy in conjunction with anti-OX40 mAb would synergize to augment anti-tumor immunity in vivo was evaluated. First, in vitro evaluation was made as to whether the IL-2 stimulation was capable of up-regulating OX40 on CD8⁺ T cells in tumor-bearing mice. IL-2 was provided via cytokine/mAb complexes (IL-2c) in order to minimize the deleterious side-effects associated with systemic rIL-2 therapy (Boyman 0, et al. Science 2006; 311: 1924-7; Krieg C, et al. Proc Natl Acad Sci USA 2010; 107: 11906-11).

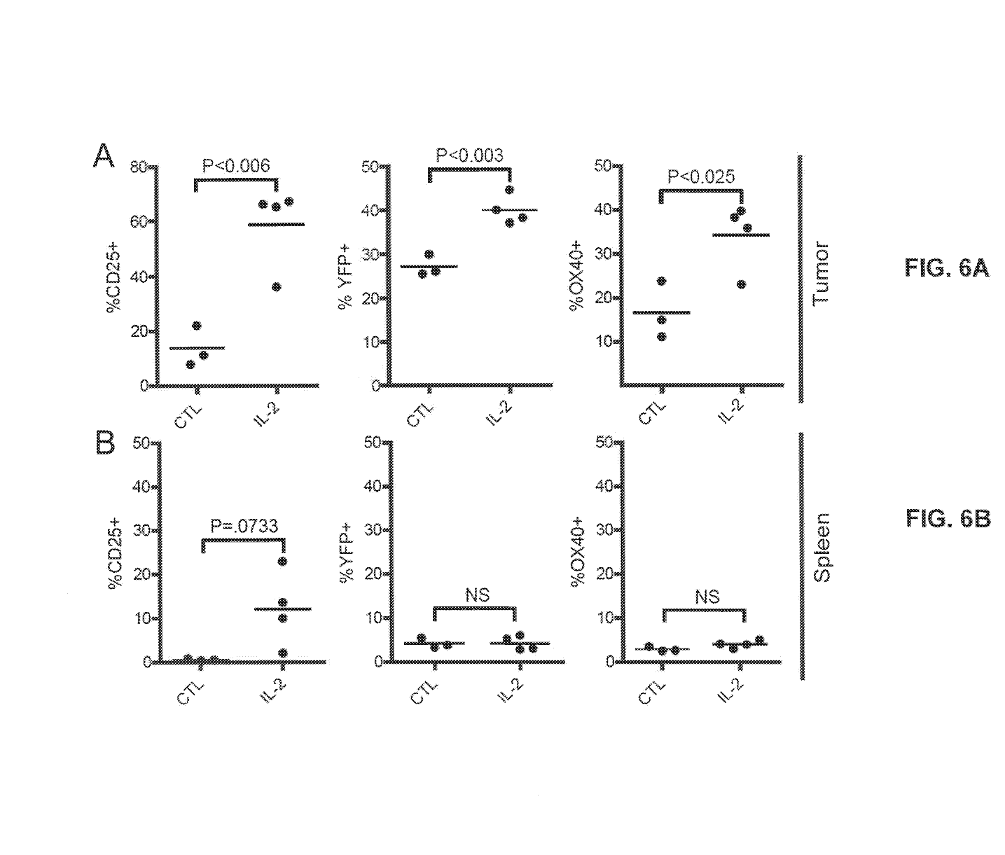

Since OX40 expression is often difficult to detect on CD8⁺ T cells stimulated in vivo, an OX40-cre x ROSA-YFP reporter mouse model was utilized (Srinivas S, et al. BMC Dev Biol 2001; 1: 4; Klinger M, et al. J Immunol 2009; 182: 4581-9) to identify OX40-expressing CD8⁺ T cells present at the tumor site or in the spleen of tumor-bearing hosts. C57BL/6 OX40-cre x ROSA-YFP reporter mice received 1×10⁶ MCA-205 sarcoma tumor cells (day 0) and two weeks later, the tumor-bearing mice were treated with IL-2 cytokine/mAb complexes (day 14, 15). Twenty four hours later (day 16 post-tumor inoculation) the extent of CD25, YFP (OX40 reporter), and OX40 expression on CD8⁺ T cells isolated from the tumor and spleen were assessed by flow cytometry. IL-2 treatment significantly enhanced CD25 and OX40 expression on CD8⁺ T cells localized in the tumor (FIG. 6A), while no significant differences were detected on CD8⁺ T cells in the spleen (FIG. 6B).

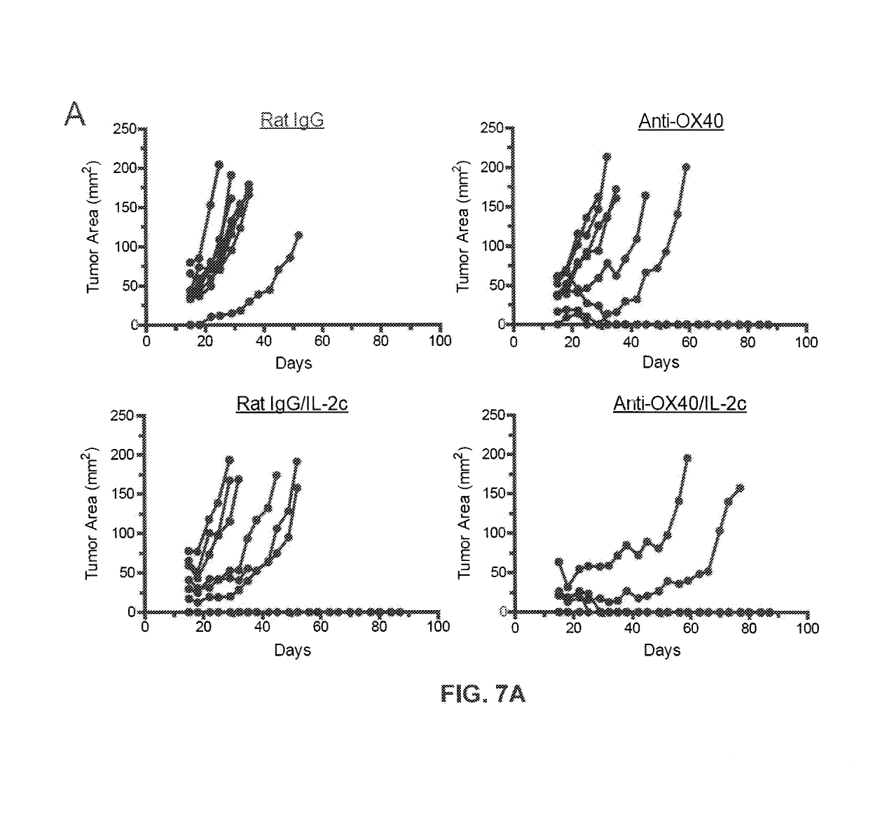

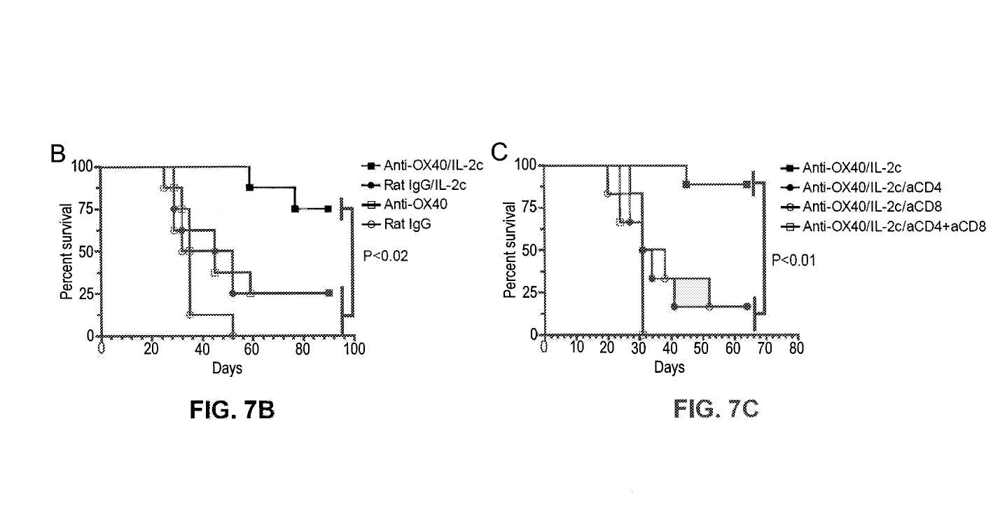

Next, the extent to which combined anti-OX40/IL-2 therapy would affect tumor growth and boost tumor immunotherapy was tested. Wild-type mice received 1×10⁶ MCA-205 sarcoma tumor cells (n=8/group). Tumor-bearing mice were treated with anti-OX40 or rat IgG Ab (days 10, 14) along with IL-2 cytokine/mAb (IL-2c) complexes (days 10-13) and the extent of tumor growth and survival of tumor-bearing mice were assessed. The results are shown in FIG. 7A and FIG. 7B. Tumor immunotherapy with combined anti-OX40/IL-2c significantly boosted tumor regression and survival compared to either treatment alone (FIGS. 7A and 7B, respectively). To determine the on-target effects of dual anti-OX40/IL-2c therapy, CD4⁺ and/or CD8⁺ T cells were depleted from cohorts of tumor-bearing mice prior to providing anti-OX40/IL-2c therapy. Further groups of MCA-205 tumor-bearing mice received no treatment (n=9), anti-CD4 (n=6), anti-CD8 (n=6), or anti-CD4+ anti-CD8 (n=3) (200 μg/dose) 9, 17, and 24 days post-tumor implantation. Mice were then treated with anti-OX40 (days 10, 14) and IL-2c (days 10-13) and the extent of survival of tumor-bearing mice was assessed. The results are shown in FIG. 7C. Depletion of either CD4⁺ or CD8⁺ T cell subsets prior to anti-OX40/IL-2c therapy abrogated the anti-tumor efficacy of the treatment (FIG. 7C).

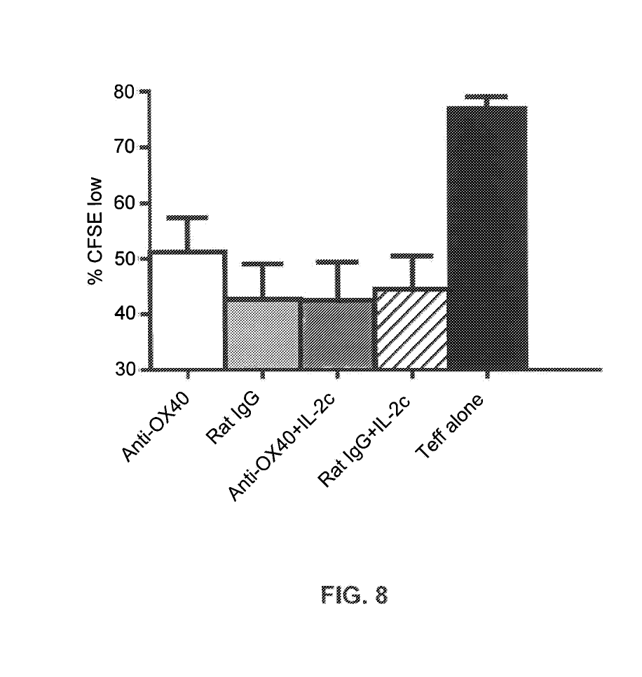

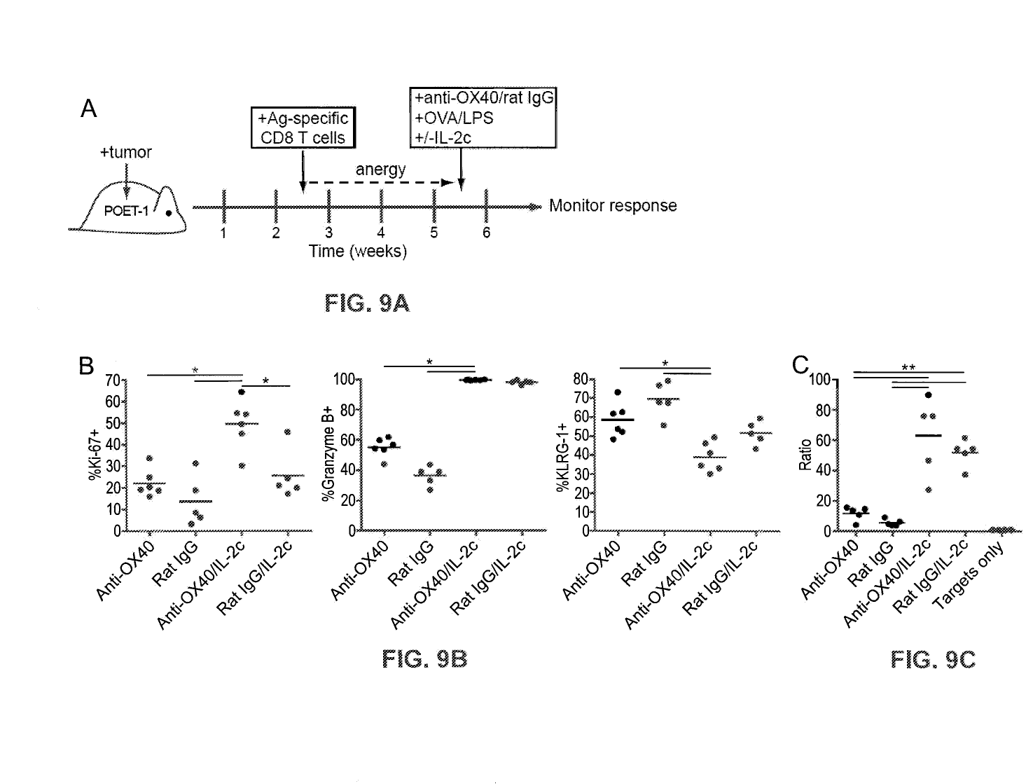

Additional studies were carried out to determine the effect of OX40 agonist/IL-2 treatment on the suppressive activity of Treg cells. Wild-type mice received 1×10⁶ MCA-205 sarcoma tumor cells (n=2-3/group). Tumor-bearing mice were treated with anti-OX40 or rat IgG Ab (days 10, 14) along with IL-2 cytokine/mAb complexes (days 10-13). On day 21, Treg were isolated from the spleens of tumor-bearing hosts and co-cultured with naïve CFSE-labeled responder CD8⁺ T cells. Cells were harvested 96 hours later and the extent of CFSE dilution in the CD8⁺ responder cells was determined by flow cytometry. The results are shown in FIG. 8. The results showed that combined anti-OX40/IL-2c therapy did not affect the suppressive activity of CD4⁺ CD25⁺ regulatory T cells, demonstrating that effector CD4⁺ and CD8⁺ T cells are required for promoting tumor regression and enhanced long-term survival following dual anti-OX40/I-2c immunotherapy.

This example shows that treatment with an agonist anti-OX40 inAb in conjunction with IL-2 can synergize to augment tumor immunotherapy. Combined anti-OX40/IL-2c therapy significantly enhanced tumor regression (FIG. 7A) and enhanced the survival of tumor-bearing hosts (FIG. 7B). The efficacy of dual anti-OX40/I-2c therapy required the presence of effector CD4⁺ and CD8⁺ T cells in the tumor-bearing host as depletion of either subset abrogated its effects (FIG. 7C), while Treg function remained unchanged (FIG. 8).

### Example 5

### Dual Anti-OX40/I-2c Therapy Reverses CD8 T Cell Anergy and Increases the Survival of Mice with Long-Term Well-Established Tumors

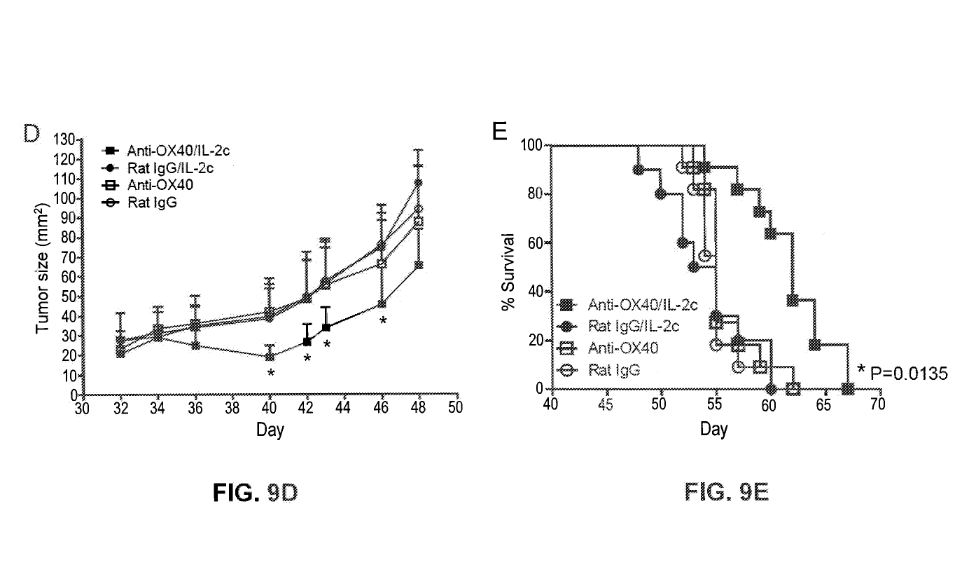

Since tumor-induced T cell anergy is an important barrier that limits the generation of potent anti-tumor immunity (Rabinovich G A, et al. Annu Rev Immunol 2007; 25: 267-96), this example investigates whether OX40 ligation in the presence of TCR/IL-2c signaling can restore the function of anergic CD8 T cells in tumor-bearing hosts. The model system used is shown in FIG. 9A. TRAMP-C1-mOVA expressing (TC1-mOVA) prostate tumor cells (2.5×10⁶ cells/mouse) were implanted in male POET-1 transgenic mice, in which prostate-specific expression of membrane-bound OVA (mOVA) is driven in an androgen-dependent manner under the control of the rat probasin promoter (Lees J R, et al. Immunology 2006; 117: 248-61; Lees J R, et al. Prostate 2006; 66: 578-90. Twenty days later, tumor-bearing mice (˜50 mm² tumors) received 5×10⁵ adoptively-transferred naïve OT-I T cells. Previous studies have shown that these tamor-reactive donor CD8 T cells become anergized in vivo Redmond W L, et al. Eur J Immunol 2009; 39: 2184-94. Seventeen days alter T cell adoptive transfer (37 days post-tumor inoculation), the anergic donor OT-I T cells were re-stimulated with anti-OX40 or control (rat IgG) Ab (days 37-38), 500 μs OVA (day 37), 10 μg LPS (day 37), +/−IL-2 cytokine/mAb complexes (days 37-44). This model allowed tracking of the response of antigen-specific CD8⁺ T cells against a surrogate tumor-associated antigen. The mice were given Ag/TLR ligand (LPS) to provide a source of TCR stimulation.

Seven days after the initial dose of Ag/anti-OX40 the extent of Ki-67 (proliferation), granzyme B, and KLRG-1 expression on the donor OT-I T cells in the peripheral blood were determined by flow cytometry. Dual anti-OX40/IL-2c therapy significantly increased the proliferative response (Ki-67) and differentiation (GrzB) of the donor cells as compared to controls (FIG. 9B).

Further analysis revealed that the majority of cells receiving dual anti-OX40/IL-2c therapy exhibited a unique phenotype characterized by limited expression of the killer cell lectin-like receptor G1 (KLRG1) (FIG. 9B), which is typically highly expressed on terminally differentiated T cells that exhibit poor long-term survival (Sarkar 5, et al. J Exp Med 2008; 205: 625-40; Joshi N S, et al. Immunity 2007; 27: 281-95; Voehringer D, et al. Blood 2002; 100: 3698-702).

To determine whether dual anti-OX40/IL-2c therapy enhanced CD8⁺ T cell cytolytic activity, an in vivo cytolytic assay was performed. Cohorts of tumor-bearing POET-1 transgenic mice prepared and treated according to the model system show in FIG. 9A, and then seven days later cognate OVA peptide-pulsed (CFSE^{high}) and control HA peptide-pulsed (CFSE^{low}) target cells were mixed at a 1:1 ratio and then injected into recipient mice. Four hours later, spleens were harvested and the ratio of % CFSEI^{low}/% CFSE^{high} target cells from individual mice (n=5/group) was determined by flow cytometry. The results are shown in FIG. 9C. Anti-OX40/IL-2c therapy led to a statistically significant increase in cytolytic activity as compared to anti-OX40 or rat IgG-treated controls and dual anti-OX40/IL-2c treated cells trended towards increased cytolytic activity as compared to IL-2c treatment alone.

Finally, the extent to which dual anti-OX40/IL-2c therapy affected tumor regression in mice with long-term well-established tumors (>40 days post-tumor implantation) was examined. The extent of tumor growth (mean+/−SD; n=5/group) and survival (n=11/group) is shown in FIG. 9D, and FIG. 9E, respectively. These data revealed that combined anti-OX40/I-2c therapy significantly enhanced tumor regression at several time points post-treatment (FIG. 9D) and also enhanced the survival of the tumor-bearing mice (FIG. 9E). This reflected a unique property of anti-OX40/IL-2 immunotherapy as treatment with anti-OX40/IL-4c or anti-OX40/IL-15c did not affect tumor growth or survival (data not shown). Together, these studies demonstrate that combined anti-OX40/IL-2c therapy can boost tumor immunotherapy by restoring the function of anergic tumor-reactive CD8⁺ T cells in vivo.

Mechanistic studies revealed that dual anti-OX40/IL-2c therapy significantly increased the proliferation (Ki-67) and differentiation (granzyme B) of anergic tumor-associated Ag-specific CD8⁺ T cells, while reducing their expression of the senescence-associated molecule KLRG1 (FIG. 9B). Although dual anti-OX40/IL-2c therapy and IL-2c treatment alone were both associated with increased cytolytic activity by the anergic CD8⁺ T cells (FIG. 9C), only dual therapy led to increased anti-tumor activity in vivo as shown by increased tumor regression and survival of mice harboring long-term well established (>5 wks) tumors (FIGS. 9D, 9E, respectively).

The present disclosure sets forth one or more but not all exemplary embodiments of the present invention as contemplated by the inventor(s), and thus is not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

1. A method of treating cancer, comprising administering to a subject in need of treatment an OX40 agonist and a common gamma chain (γc) cytokine or an active fragment, variant, analog, or derivative thereof.

2. The method of claim 1, wherein the administration stimulates T-lymphocyte-mediated anti-cancer immunity to a greater extent than the OX40 agonist or γc cytokine alone.

3. The method of claim 1, wherein the administration can restore the function, proliferation, or differentiation of anergic tumor-reactive CD8⁺ T-lymphocytes.

4. (canceled)

5. (canceled)

6. A method of enhancing the effect of an OX40 agonist on T-lymphocyte mediated cancer immunotherapy, comprising contacting T Cell Receptor (TCR)-stimulated T-lymphocytes with an OX40 agonist in combination with a γc cytokine, or an active fragment, variant, analog, or derivative thereof.

7. The method of claim 6, further comprising stimulating T-lymphocytes via TCR ligation.

8. The method of claim 6, wherein the cancer immunotherapy requires both CD4⁺ T-lymphocytes and CD8⁺ T-lymphocytes.

9. (canceled)

10. The method of claim 6, wherein the contacting can restore the function of anergic tumor-reactive CD8⁺ T cells.

11. A method of enhancing OX40 agonist-mediated augmentation of T-lymphocyte proliferation in response to TCR stimulation, comprising contacting TCR-stimulated T-lymphocytes with an OX40 agonist in combination with a γc cytokine, or an active fragment, variant, analog, or derivative thereof.

12. The method of claim 11, further comprising stimulating T-lymphocytes via TCR ligation.

13. The method of claim 11, wherein T-lymphocyte differentiation is enhanced.

14. The method of claim 7, wherein TCR ligation is accomplished through contacting the T-lymphocytes with an antigen/MHC complex.

15. The method of claim 14, wherein the antigen is a cancer cell-specific antigen.

16. The method of claim 7, wherein the TCR ligation is accomplished through contacting the T-lymphocytes with an anti-CD3 antibody.

17. (canceled)

18. The method of claim 16, further comprising contacting the T-lymphocytes with an anti-CD28 antibody.

19. (canceled)

20. (canceled)

21. The method of claim 1, wherein the γc cytokine is selected from the group consisting of IL-2, IL-4, IL-7, IL-21, any active fragment, variant, analog or derivative thereof and a combination thereof.

22. (canceled)

23. The method of claim 1, wherein the γc cytokine upregulates OX40 expression in the T-lymphocytes.

24. (canceled)

25. (canceled)

26. (canceled)

27. (canceled)

28. The method of claim 1, wherein the OX40 agonist is an antibody which specifically binds to OX40, or an antigen-binding fragment thereof.

29. (canceled)

30. (canceled)

31. (canceled)

32. (canceled)

33. (canceled)

34. (canceled)

35. (canceled)

36. (canceled)

37. (canceled)

38. The method of claim 28, wherein the antibody or antigen-binding fragment thereof binds to the same OX40 epitope as mAb 9B12.

39. The method of claim 1, wherein the OX40 agonist is an OX40 ligand or OX40-binding fragment thereof.

40. (canceled)

41. (canceled)

42. The method of claim 1, wherein the OX40 agonist is a fusion polypeptide comprising in an N-terminal to C-terminal direction:
an immunoglobulin domain, wherein the immunoglobulin domain comprises an Fc domain;
a trimerization domain, wherein the trimerization domain comprises a coiled coil trimerization domain; and
a receptor binding domain, wherein the receptor binding domain is an OX40 receptor binding domain,
and wherein the fusion polypeptide self-assembles into a trimeric fusion protein.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The method of claim 1, wherein the administration retards, stalls or decreases growth of a long-term established tumor or metastasis thereof or results in retarded or no increase in tumor or metastatic growth, stabilization of disease, or prolonged survival of the subject.

56. The method of claim 1, wherein the γc cytokine is administered to the subject prior to, simultaneously with, or after the, administration of the OX40 agonist.

57. (canceled)

58. (canceled)

59. The method of claim 21, wherein the IL-2 is aldesleukin, BAY 50-4798, NHS-EMD 521873, an IL-2/anti-IL-2 complex, or any combination thereof.
